(19) United States
(12) Patent Application Publication
(10) Pub. No.: US 2008/0102520 A1
(43) Pub. Date: May 1, 2008

(54) Novel human membrane proteins and polynucleotides encoding the same

(76) Inventors: Alejandro Abuin, The Woodlands, TX (US); Michael B. Burnett, Houston, TX (US); Emily B. Cullinan, The Woodlands, TX (US); Gregory Donoho, Indianapolis, IN (US); Dorit B. Donoviel, The Woodlands, TX (US); Carl Johan Friddle, The Woodlands, TX (US); Glenn Friedrich, Houston, TX (US); Brenda Gerhardt, Spring, TX (US); Erin Hilbun, Denton, TX (US); Yi Hu, Spring, TX (US); Brian Mathur, Nashville, TN (US); Maricar Miranda, The Woodlands, TX (US); Michael C. Nehls, Stockdorf (DE); Boris Nepomnichy, Dickinson, TX (US); David George Potter, The Woodlands, TX (US); David Reed Powell, Houston, TX (US); Arthur T. Sands, The Woodlands, TX (US); John Scoville, Pearland, TX (US); Hong Sun, Bellevue, WA (US); C. Alexander Turner, The Woodlands, TX (US); D. Wade Walke, Spring, TX (US); Frank Wattler, Muensing (DE); Nathaniel L. Wilganowski, Spring, TX (US); Xuanchuan Yu, Conroe, TX (US); Brian Zambrowicz, The Woodlands, TX (US)

Correspondence Address: LEXICON PHARMACEUTICALS, INC., 8800 TECHNOLOGY FOREST PLACE, THE WOODLANDS, TX, 77381-1160, US

(21) Appl. No.: 11/356,592

(22) Filed: Feb. 17, 2006

Related U.S. Application Data
(63) Continuation-in-part of application No. 11/196,524, filed on Aug. 3, 2005, now abandoned.
Application No. 11/196,524 is a division of application No. 10/165,813, filed on Jun. 7, 2002, now Pat. No. 6,987,178.
Application No. 10/165,813 is a continuation of application No. 09/501,558, filed on Feb. 9, 2000, now Pat. No. 6,403,784.
(60) Provisional application No. 60/158,458, filed on Oct. 8, 1999.
(60) Provisional application No. 60/119,228, filed on Feb. 9, 1999.

(51) Int. Cl. C12N 15/64; C07H 21/04; C07K 16/44

(52) U.S. Cl. 435/320.1; 530/387.9; 536/23.1; 536/24.5

(57) ABSTRACT

Novel human polynucleotide and polypeptide sequences are disclosed that can be used in therapeutic, diagnostic, and pharmacogenomic applications.

### 1.0 CROSS-REFERENCE TO RELATED APPLICATIONS

The present application is a continuation-in-part of: co-pending U.S. application Ser. No. 11/196,524, filed on Aug. 3, 2005, which is a divisional of U.S. application Ser. No. 10/165,813, filed on Jun. 7, 2002, which issued as U.S. Pat. No. 6,987,178 B2 on Jan. 17, 2006, which is a continuation of U.S. application Ser. No. 09/501,558, filed on Feb. 9, 2000, which issued as U.S. Pat. No. 6,403,784 B1 on Jun. 11, 2002, which claims the benefit of U.S. Provisional Application Nos. 60/158,458, filed on Oct. 8, 1999, and 60/119,228, filed Feb. 9, 1999; co-pending U.S. application Ser. No. 10/900,742, filed on Jul. 28, 2004, which is a continuation of U.S. application Ser. No. 09/710,098, filed on Nov. 10, 2000, abandoned, which claims the benefit of U.S. Provisional Application No. 60/165,263, filed on Nov. 12, 1999; co-pending U.S. application Ser. No. 11/023,060, filed on Dec. 27, 2004, which is a continuation of U.S. application Ser. No. 09/714,882, filed on Nov. 16, 2000, abandoned, which claims the benefit of U.S. Provisional Application No. 60/165,959, filed on Nov. 17, 1999; co-pending U.S. application Ser. No. 11/179,018, filed on Jul. 11, 2005, which is a continuation of U.S. application Ser. No. 09/735,712, filed on Dec. 12, 2000, abandoned, which claims the benefit of U.S. Provisional Application No. 60/171,567, filed on Dec. 22, 1999; co-pending U.S. application Ser. No. 10/942,745, filed on Sep. 16, 2004, which is a continuation of U.S. application Ser. No. 09/771,961, filed on Jan. 29, 2001, abandoned, which claims the benefit of U.S. Provisional Application No. 60/179,001, filed on Jan. 28, 2000; co-pending U.S. application Ser. No. 10/999,233, filed on Nov. 29, 2004, which is a continuation of U.S. application Ser. No. 09/770,643, filed on Jan. 26, 2001, abandoned, which claims the benefit of U.S. Provisional Application Nos. 60/199,513, filed on Apr. 25, 2000, and 60/178,557, filed on Jan. 26, 2000; co-pending U.S. application Ser. No. 11/188,353, filed on Jul. 25, 2005, which is a continuation of U.S. application Ser. No. 10/436,356, filed on May 12, 2003, abandoned, which is a continuation of U.S. application Ser. No. 09/765,069, filed on Jan. 18, 2001, which issued as U.S. Pat. No. 6,586,582 B2 on Jul. 1, 2003, which claims the benefit of U.S. Provisional Application No. 60/176,692, filed on Jan. 18, 2000; co-pending U.S. application Ser. No. 10/862,060, filed on Jun. 4, 2004, which is a continuation of U.S. application Ser. No. 09/822,807, filed on Mar. 30, 2001, abandoned, which claims the benefit of U.S. Provisional Application No. 60/193,336, filed on Mar. 30, 2000; co-pending U.S. application Ser. No. 10/843,132, filed on May 11, 2004, which is a continuation of U.S. application Ser. No. 09/870,113, filed on May 30, 2001, which issued as U.S. Pat. No. 6,790,667 B1 on Sep. 14, 2004, which claims the benefit of U.S. Provisional Application No. 60/207,933, filed on May 30, 2000; co-pending U.S. application Ser. No. 10/901,909, filed on Jul. 29, 2004, which is a continuation of U.S. application Ser. No. 09/876,858, filed on Jun. 7, 2001, abandoned, which claims the benefit of U.S. Provisional Application No. 60/210,271, filed on Jun. 8, 2000; co-pending U.S. application Ser. No. 09/893,321, filed on Jun. 27, 2001, which claims the benefit of U.S. Provisional Application No. 60/214,083, filed on Jun. 27, 2000; co-pending U.S. application Ser. No. 10/925,362, filed on Aug. 24, 2004, which is a continuation of U.S. application Ser. No. 09/939,512, filed on Aug. 24, 2001, abandoned, which claims the benefit of U.S. Provisional Application Nos. 60/231,044, filed on Sep. 8, 2000, and 60/227,754, filed on Aug. 24, 2000; co-pending U.S. application Ser. No. 10/798,721, filed on Mar. 11, 2004, which is a continuation of U.S. application Ser. No. 09/969,532, filed on Oct. 2, 2001, which issued as U.S. Pat. No. 6,777,232 B1 on Aug. 17, 2004, which claims the benefit of U.S. Provisional Application No. 60/237,280, filed on Oct. 2, 2000; co-pending U.S. application Ser. No. 11/264,289, filed on Oct. 31, 2005, which is a continuation of co-pending U.S. application Ser. No. 09/981,318, filed on Oct. 17, 2001, which claims the benefit of U.S. Provisional Application No. 60/241,194, filed on Oct. 17, 2000; co-pending U.S. application Ser. No. 10/917,242, filed on Aug. 12, 2004, which is a divisional of U.S. application Ser. No. 10/025,225, filed on Dec. 19, 2001, which issued as U.S. Pat. No. 6,852,844 B1 on Feb. 8, 2005, which claims the benefit of U.S. Provisional Application No. 60/257,257, filed on Dec. 20, 2000; co-pending U.S. application Ser. No. 10/885,484, filed on Jul. 6, 2004, which is a continuation of U.S. application Ser. No. 10/067,162, filed on Feb. 4, 2002, abandoned, which claims the benefit of U.S. Provisional Application No. 60/266,739, filed on Feb. 6, 2001; co-pending U.S. application Ser. No. 10/859,039, filed on Jun. 1, 2004, which is a continuation of U.S. application Ser. No. 10/079,267, filed on Feb. 19, 2002, abandoned, which claims the benefit of U.S. Provisional Application No. 60/271,335, filed on Feb. 23, 2001; co-pending U.S. application Ser. No. 10/090,466, filed on Mar. 1, 2002, which claims the benefit of U.S. Provisional Application No. 60/274,961, filed on Mar. 12, 2001; co-pending U.S. application Ser. No. 11/230,321, filed on Sep. 19, 2005, which is a divisional of U.S. application Ser. No. 10/094,162, filed on Mar. 6, 2002, which issued as U.S. Pat. No. 6,994,995 B1 on Feb. 7, 2006, which claims the benefit of U.S. Provisional Application No. 60/276,594, filed on Mar. 16, 2001; co-pending U.S. application Ser. No. 11/135,604, filed on May 23, 2005, which is a continuation of U.S. application Ser. No. 10/132,089, filed on Apr. 24, 2002, abandoned, which claims the benefit of U.S. Provisional Application No. 60/287,641, filed on Apr. 30, 2001; co-pending U.S. application Ser. No. 10/990,763, filed on Nov. 17, 2004, which is a continuation of U.S. application Ser. No. 10/123,962, filed on Apr. 16, 2002, abandoned, which claims the benefit of U.S. Provisional Application No. 60/286,141, filed on Apr. 24, 2001; co-pending U.S. application Ser. No. 10/160,567, filed on May 31, 2002, which claims the benefit of U.S. Provisional Application No. 60/294,882, filed on May 31, 2001; co-pending U.S. application Ser. No. 10/223,667, filed on Aug. 16, 2002, which claims the benefit of U.S. Provisional Application No. 60/312,939, filed on Aug. 16, 2001; and co-pending U.S. application Ser. No. 10/225,544, filed on Aug. 21, 2002, which claims the benefit of U.S. Provisional Application No. 60/313,876, filed on Aug. 21, 2001; each of which is herein incorporated by reference in its entirety.

### 2.0 CROSS-REFERENCE TO SEQUENCE LISTING SUBMITTED ON COMPACT DISC

The present application contains a Sequence Listing of SEQ ID NOS:1-270, in file “FINALseqlist.txt” (2,086,912 bytes), created on Feb. 16, 2006, submitted herewith on duplicate compact disc (Copy 1 and Copy 2), which is herein incorporated by reference in its entirety.

### 3.0 INTRODUCTION

The present invention relates to the discovery, identification, and characterization of novel human polynucleotides encoding proteins that share sequence similarity with mammalian membrane proteins. The invention encompasses the described polynucleotides, host cell expression systems, the encoded proteins, fusion proteins, polypeptides and peptides, antibodies to the encoded proteins and peptides, and genetically engineered animals that either lack or overexpress the disclosed polynucleotides, antagonists and agonists of the proteins, and other compounds that modulate the expression or activity of the proteins encoded by the disclosed polynucleotides, which can be used for diagnosis, drug screening, clinical trial monitoring, the treatment of diseases and disorders, and cosmetic or nutriceutical applications.

### 4.0 BACKGROUND OF THE INVENTION

In addition to providing the structural and mechanical scaffolding for cells and tissues, membrane proteins also mediate a variety of regulatory and housekeeping functions within the cell, and can also serve as receptors, recognition or cell surface markers, mediate cell-cell interactions, mediate signal transduction, and can mediate or facilitate the passage of materials across the lipid bilayer.

Many proteins present in the mitochondria are encoded within the nucleus. Uncoupling proteins (UCPs) are one example of such nuclear encoded mitochondrial proteins. In the mitochondria, nuclear encoded mitochondrial proteins are involved in, or regulate, the gradient that drives energy production in the cell/body. UCPs uncouple this gradient. As such, nuclear encoded mitochondrial proteins, including, but not limited to, UCPs, effectively modulate the efficiency of energy production in the body, and hence body metabolism. Given the role of nuclear encoded mitochondrial proteins, including UCPs, in the body, they are thought to be important targets for the study of obesity, cachexia, thermogenesis, and other metabolically related physiological functions, diseases, and disorders.

Transporter proteins are integral membrane proteins that mediate or facilitate the passage of materials across the lipid bilayer. Given that the transport of materials across the membrane can play an important physiological role, transporter proteins are good drug targets. Additionally, one of the mechanisms of drug resistance involves diseased cells using cellular transporter systems to export chemotherapeutic agents from the cell. Such mechanisms are particularly relevant to cells manifesting resistance to a multiplicity of drugs.

SEL-1 proteins are negative regulators of Notch family receptors. Notch receptors and their associated signaling pathways have been associated with development, apoptosis, neuron growth and maintenance. Genetic alterations in Notch receptors and their ligands have been associated with multiple human processes and disorders, such as diabetes, cancer (inter alia pancreatic cancer and insulinomas), stroke, Alzheimer's, and other neurodegenerative diseases, cholesterol and fat metabolism (HMG CoA reductase degradation), blood pressure abnormalities, coronary artery disease, and immunity (see, e.g., PCT Patent Application Serial No. PCT/CA98/01058, Publication No. WO 99/27088).

Neurexins have been associated with, inter alia, mediating neural processes, seizures, signaling, exocytosis, cancer, and development. Neurexins can also serve as receptors for latrotoxins. Semaphorins are proteins that have been implicated in a number of biological processes and anomalies such as neural development, paralysis, and axon guidance. Cadherin proteins are membrane proteins that have been linked to a variety of biological processes varying from development, tumor suppression, neural function, and cell communication. Synaptotagmins are a family of proteins that have been implicated in membrane traffic and calcium dependent neuroexocytosis.

The kidney is a primary organ for the ultra filtration of blood and plasma in the human body. To effect such ultra filtration, the kidney employs a plurality of glomeruli that each in turn incorporate a fenestrated endothelial layer over a basement membrane. The glomeruli collectively constitute a size-selective molecular sieve that can respond to or regulate a variety of chemical and physiological equilibria in the body. Given the critical importance of kidney function in mammals, the kidney, and its related disorders has been subject to intensive medical scrutiny.

Sensory receptor proteins are typically membrane proteins that interact with ligands or stimuli and mediate signal transduction. The IgE receptor plays a role in the activation and release of agents that mediate a variety of allergic and inflammatory reactions. GABA receptors bind potent inhibitory neurotransmitters and this interaction serves as a target for a variety of pharmaceutically active agents such as benzodiazepines, barbiturates, and alcohol.

Therefore, membrane proteins constitute ideal targets for drug intervention and for the design of therapeutic agents.

### 5.0 SUMMARY OF THE INVENTION

The present invention relates to the discovery, identification, and characterization of nucleotides that encode novel human membrane proteins and the corresponding amino acid sequences of these proteins. The novel human membrane proteins, described for the first time herein, share structural similarity with: mammalian uncoupling proteins and brain mitochondrial carrier proteins (SEQ ID NOS:1-4); mammalian class III MHC membrane receptor proteins, and membrane receptors such as, but not limited to, MHC and HLA proteins (SEQ ID NOS:5-8); animal Notch ligands, and particularly SEL-1 (SEQ ID NOS:9-22); membrane receptors such as, but not limited to, the IgE receptor and mammalian CD20 (SEQ ID NOS:23-31); membrane receptors such as, but not limited to, mammalian CD82 and CD37 (SEQ ID NOS:32-35); animal neurexin proteins (including secreted types) and contactin associated proteins (SEQ ID NOS:36-62); mammalian gamma-amino butyric acid (GABA) receptor subunits, and particularly rho 3 subunits of the GABA receptor (SEQ ID NOS:63-74); mammalian proteins that are induced in response to viral infection and hormone or cytokine stimulation, and proteins expressed in the central nervous system (SEQ ID NOS:75-91); mammalian mitochondrial proteins that are encoded in the nucleus, such as mitochondrial solute carriers, RNA splicing proteins, uncoupling proteins, and mitochondrial carrier proteins (SEQ ID NOS:92-103); human nephrin, and animal titin, robo, and irregular chiasm C-roughest precursor (SEQ ID NOS:104-107); human and other mammalian GABA receptor subunits, and particularly GABA A receptor gamma-1, -2, -3, -4, -5, and -6 subunits (SEQ ID NOS:108-112); mammalian neurexins, agrin, notch proteins, and proteins having EGF domains (SEQ ID NOS:113-124); mammalian protein and peptide receptors, and particularly proteins of the Unc5 family, which are putative netrin receptors (SEQ ID NOS:125-157); animal semaphorin proteins (SEQ ID NOS:158-192); animal cadherin and protocadherin proteins, especially the protocadherin FAT (SEQ ID NOS:193-200); mammalian membrane proteins, and particularly ion channel/transporter proteins of the phospholemman family (SEQ ID NOS:201-203); mammalian membrane proteins (SEQ ID NOS:204 and 205); mammalian proteins having structural domains in common with proteins of the immunoglobulin (Ig) super family, and proteins of the Ig Fc receptor family, which are often found on the cell surface and can be exploited by human pathogens to gain entry to the cell (SEQ ID NOS:206-214); mammalian proteins having structural domains in common with proteins of the dectin family, which are typically integral membrane proteins exhibiting pectin/ligand binding domains (SEQ ID NOS:215-218); animal synaptotagmins, in particular synaptotagmin 2, a calcium binding/sensing protein that is present on synaptic vesicles (SEQ ID NOS:219-221); mammalian transporters, such as those involved in multi-drug resistance (MDR), and particularly nuclear importins, which allow selective protein import into the cell nucleus (SEQ ID NOS:222-228); mammalian proteins of the epidermal growth factor (EGF) family, stabilin, and notch proteins (SEQ ID NOS:229-233); mammalian proteins having structural domains in common with animal tectorin and uromodulin proteins (SEQ ID NOS:234-236); mammalian proteins of the MUNC family (mammalian homologs of worm unc proteins, which are essential for presynaptic function in worms), and particularly MUNC-13, which have been implicated in neural development and function (SEQ ID NOS:237-240); animal proteins of the cadherin family, and closely resemble the Drosophila tumor suppressor FAT (SEQ ID NOS:241-262); and mammalian growth factor receptors and plexins (SEQ ID NOS:263-270).

The novel human nucleic acid sequences described herein (SEQ ID NOS:1, 3, 5, 7, 9, 11, 13, 15, 17, 19, 21, 23, 25, 27, 29, 32, 34, 36, 38, 40, 42, 44, 46, 48, 50, 52, 54, 56, 58, 60, 63, 65, 67, 69, 71, 73, 75, 77, 79, 81, 83, 85, 87, 89, 92, 94, 96, 98, 100, 102, 104, 106, 108, 110, 113, 115, 118, 120, 122, 125, 127, 129, 131, 133, 135, 137, 139, 141, 143, 145, 147, 149, 151, 153, 155, 158, 160, 162, 164, 166, 168, 170, 172, 174, 176, 178, 180, 182, 184, 186, 188, 190, 193, 195, 197, 199, 201, 204, 206, 208, 210, 212, 215, 217, 219, 222, 224, 226, 229, 231, 234, 237, 239, 241, 243, 245, 247, 249, 251, 253, 255, 257, 259, 261, 263, 265, 267, and 269) encode alternative proteins/open reading frames (ORFs) of 291, 293, 585, 584, 689, 688, 590, 418, 499, 576, 575, 199, 81, 138, 149, 248, 211, 1307, 1259, 35, 250, 279, 582, 534, 745, 697, 839, 791, 1298, 1175, 467, 392, 180, 420, 345, 133, 513, 690, 477, 654, 379, 556, 343, 520, 364, 193, 230, 265, 94, 131, 605, 403, 465, 256, 1009, 152, 285, 154, 134, 577, 566, 563, 552, 911, 900, 897, 886, 346, 335, 332, 321, 680, 669, 666, 655, 767, 1047, 1062, 838, 1150, 863, 1158, 890, 1185, 714, 994, 1009, 785, 1097, 810, 1105, 1132, 4589, 3852, 4585, 4588, 95, 504, 426, 462, 203, 239, 213, 182, 419, 516, 532, 537, 1986, 2017, 415, 1971, 764, 885, 854, 231, 613, 860, 693, 829, 588, 443, 662, 668, 3035, 2033, 699, and 1776 amino acids in length (SEQ ID NOS:2, 4, 6, 8, 10, 12, 14, 16, 18, 20, 22, 24, 26, 28, 30, 33, 35, 37, 39, 41, 43, 45, 47, 49, 51, 53, 55, 57, 59, 61, 64, 66, 68, 70, 72, 74, 76, 78, 80, 82, 84, 86, 88, 90, 93, 95, 97, 99, 101, 103, 105, 107, 109, 111, 114, 116, 119, 121, 123, 126, 128, 130, 132, 134, 136, 138, 140, 142, 144, 146, 148, 150, 152, 154, 156, 159, 161, 163, 165, 167, 169, 171, 173, 175, 177, 179, 181, 183, 185, 187, 189, 191, 194, 196, 198, 200, 202, 205, 207, 209, 211, 213, 216, 218, 220, 223, 225, 227, 230, 232, 235, 238, 240, 242, 244, 246, 248, 250, 252, 254, 256, 258, 260, 262, 264, 266, 268, and 270, respectively). SEQ ID NOS:31, 62, 91, 112, 117, 124, 157, 192, 203, 214, 221, 228, 233, and 236 describe full length ORFs, as well as flanking 5′ and 3′ sequences.

The invention also encompasses agonists and antagonists of the described membrane proteins, including small molecules, large molecules, mutant membrane proteins, or portions thereof, that compete with native membrane proteins, peptides, and antibodies, as well as nucleotide sequences that can be used to inhibit the expression of the described membrane proteins (e.g., antisense and ribozyme molecules, and open reading frame or regulatory sequence replacement constructs) or to enhance the expression of the described membrane proteins (e.g., expression constructs that place the described polynucleotide under the control of a strong promoter system), and transgenic animals that express a membrane protein sequence, or “knock-outs” (which can be conditional) that do not express a functional membrane protein.

Murine homologs of many of the described human membrane proteins have been identified, and “knockout” ES cell lines have been produced using the methods such as those described in U.S. Pat. Nos. 6,136,566, 6,139,833, and 6,207,371, U.S. patent application Ser. No. 08/728,963, and “Mouse Mutagenesis” (Zambrowicz et al., eds., Lexicon Press, The Woodlands, Tex., 1998), and periodic updates thereof. Knock-out mice can be produced in several ways, one of which involves the use of mouse embryonic stem cell (“ES cell”) lines that contain gene trap mutations in a murine homolog of at least one of the described membrane proteins. Alternatively, such knock-out cells and animals can be produced using conventional methods for generating genetically engineered animals and cells (see, e.g., PCT Patent Application Serial No. PCT/US98/03243, Publication Number WO 98/37175). When the unique membrane protein sequences described in SEQ ID NOS:1-270 are “knocked-out” they provide a method of identifying phenotypic expression of the particular gene, as well as a method of assigning function to previously unknown genes. For example, knock-out mice corresponding to the murine homolog of SEQ ID NOS:104-107 have been produced that display profound developmental abnormalities in the kidney, and knock-out mice corresponding to the murine homolog of SEQ ID NOS:158-192 demonstrated a decrease in body-weight as compared to control wild-type mice, with no additional phenotypic alterations observed. In addition, animals in which the unique membrane protein sequences described in SEQ ID NOS:1-270 are “knocked-out” provide an unique source in which to elicit antibodies to homologous and orthologous proteins, which would have been previously viewed by the immune system as “self” and therefore would have failed to elicit significant antibody responses.

Additionally, the unique membrane protein sequences described in SEQ ID NOS:1-270 are useful for the identification of protein coding sequences, and mapping an unique gene to a particular chromosome. These sequences identify biologically verified exon splice junctions, as opposed to splice junctions that may have been bioinformatically predicted from genomic sequence alone. The sequences of the present invention are also useful as additional DNA markers for restriction fragment length polymorphism (RFLP) analysis, and in forensic biology, particularly given the presence of nucleotide polymorphisms within the described sequences.

Further, the present invention also relates to processes for identifying compounds that modulate, i.e., act as agonists or antagonists of, membrane protein expression and/or membrane protein activity that utilize purified preparations of the described membrane proteins and/or membrane protein products, or cells expressing the same. Such compounds can be used as therapeutic agents for the treatment of any of a wide variety of symptoms associated with biological disorders or imbalances.

### 6.0 BRIEF DESCRIPTION OF THE FIGURES

No Figures are required in the present invention.

### 7.0 DETAILED DESCRIPTION OF THE INVENTION

The membrane proteins described for the first time herein are novel proteins that can be found expressed in, inter alia, human cell lines, and: human cerebellum, spinal cord, thymus, spleen, lymph node, bone marrow, trachea, lung, kidney, fetal liver, prostate, testis, thyroid, salivary gland, stomach, heart, uterus, and mammary gland cells, with particularly strong expression in human kidney, adrenal gland, heart, and skeletal muscle cells (SEQ ID NOS:1-4); human kidney, bone marrow, adrenal, trachea, brain, pancreas, mammary gland, placenta, prostate, thymus, liver, lymph node, and testis cells (SEQ ID NOS:5-8); human testis cells (SEQ ID NOS:9-31); human trachea, prostate, testis, thyroid, salivary gland, small intestine, skeletal muscle, heart, uterus, mammary gland, adipose, esophagus, cervix, pericardium, hypothalamus, ovary, and fetal lung cells (SEQ ID NOS:32-35); human fetal brain, brain, cerebellum, testis, adrenal-gland, spinal cord, small intestine, and hypothalamus cells (SEQ ID NOS:36-62); human testis, brain, and adrenal gland cells (SEQ ID NOS:63-74); human fetal brain, brain, pituitary, cerebellum, spinal cord, lymph node, testis, thyroid, pancreas, and hypothalamus cells, and predominantly expressed in the central nervous system (SEQ ID NOS:75-91); human fetal brain, pituitary, trachea, lung, salivary gland, and fetal kidney cells (SEQ ID NOS:92-103); human fetal kidney and adipose cells (SEQ ID NOS:104-107); human brain, pituitary, cerebellum, lymph node, adipose, esophagus, cervix, rectum, pericardium, and hypothalamus cells (SEQ ID NOS:108-112); human spinal cord, spleen, lymph node, bone marrow, trachea, lung, kidney, fetal liver, prostate, testis, thyroid, adrenal gland, salivary gland, stomach, small intestine, colon, heart, uterus, placenta, mammary gland, adipose, esophagus, bladder, cervix, pericardium, ovary, fetal kidney, and fetal lung cells (SEQ ID NOS:113-117); human fetal brain, brain, pituitary, cerebellum, spinal cord, thymus, spleen, lymph node, bone marrow, trachea, lung, kidney, fetal liver, liver, prostate, testis, thyroid, adrenal gland, pancreas, salivary gland, stomach, small intestine, colon, skeletal muscle, heart, uterus, placenta, mammary gland, adipose, skin, esophagus, bladder, cervix, rectum, pericardium, fetal kidney, and fetal lung cells (SEQ ID NOS:118-124); human fetal brain, brain, pituitary, cerebellum, spinal cord, thymus, kidney, prostate, testis, adrenal gland, stomach, small intestine, mammary gland, esophagus, bladder, cervix, pericardium, and fetal kidney cells (SEQ ID NOS:125-157); human fetal brain, brain, pituitary, spinal cord, spleen, lymph node, bone marrow, lung, kidney, fetal liver, prostate, adrenal gland, pancreas, salivary gland, stomach, small intestine, colon, placenta, skin, bladder, cervix, pericardium, ovary, fetal lung, fetal kidney, and fetal lung cells (SEQ ID NOS:158-192); human fetal brain, brain, pituitary, cerebellum, fetal kidney, fetal lung, and 6- and 9-week embryo cells (SEQ ID NOS:193-200); human fetal brain, cerebellum, thymus, spleen, bone marrow, lymph node, trachea, kidney, fetal liver, prostate, testis, thyroid, adrenal gland, pancreas, skeletal muscle, uterus, bladder, cervix, fetal kidney, fetal lung, gall bladder, 6- and 9-week old embryo, adenocarcinoma, osteosarcoma, and embryonic carcinoma cells (SEQ ID NOS:201-203); human pituitary, spinal cord, bone marrow, lymph node, prostate, thyroid, adrenal gland, salivary gland, stomach, small intestine, skeletal muscle, uterus, placenta, mammary gland, bladder, rectum, pericardium, fetal kidney, fetal lung, tongue, 6-, 9-, and 12-week old embryo, adenocarcinoma, osteosarcoma, embryonic carcinoma, and normal umbilical vein cells (SEQ ID NOS:204 and 205); human lymph node, fetal kidney, and fetal lung cells (SEQ ID NOS:206-214); human lymph node, bone marrow, testis, fetal kidney, fetal lung, and embryonic carcinoma cells (SEQ ID NOS:215-218); human fetal brain, cerebellum, spinal cord, lymph node, testis, small intestine, esophagus, hypothalamus, ovary, fetal kidney, fetal lung, and 6- and 9-week old embryo cells (SEQ ID NOS:219-221); human fetal brain, spinal cord, thymus, spleen, lymph node, bone marrow, trachea, lung, kidney, fetal liver, liver, prostate, testis, thyroid, adrenal gland, pancreas, salivary gland, stomach, small intestine, colon, skeletal muscle, uterus, placenta, mammary gland, esophagus, bladder, cervix, rectum, pericardium, hypothalamus, ovary, fetal kidney, fetal lung, gall bladder, tongue, aorta, 6-, 9-, and 12-week embryo, osteosarcoma, and embryonic carcinoma cells (SEQ ID NOS:222-228); human spleen, lymph node, bone marrow, trachea, lung, kidney, fetal liver, liver, testis, thyroid, adrenal gland, pancreas, stomach, small intestine, colon, skeletal muscle, heart, uterus, placenta, mammary gland, adipose, esophagus, bladder, cervix, rectum, pericardium, ovary, fetal kidney, fetal lung, gall bladder, aorta, and 6-, 9-, and 12-week old embryo cells (SEQ ID NOS:229-233); human cervix, rectum, kidney, testis, fetal kidney, and fetal lung cells (SEQ ID NOS:234-236); human fetal brain, brain, cerebellum, spinal cord, lymph node, bone marrow, trachea, lung, kidney, fetal liver, prostate, testis, thyroid, adrenal gland, pancreas, salivary gland, stomach, small intestine, colon, skeletal muscle, uterus, placenta, mammary gland, adipose, esophagus, bladder, cervix, rectum, pericardium, hypothalamus, fetal kidney, fetal lung, gall bladder, tongue, aorta, 6-, 9-, and 12-week old embryo, umbilical vein, and osteosarcoma cells (SEQ ID NOS:237-240); human fetal brain, brain, cerebellum, spinal cord, lymph node, trachea, lung, kidney, fetal liver, liver, prostate, testis, thyroid, adrenal gland, stomach, small intestine, colon, uterus, placenta, mammary gland, esophagus, bladder, cervix, ovary, fetal kidney, fetal lung, 6-, 9-, and 12-week embryo, and osteosarcoma cells (SEQ ID NOS:241-262); and human lymph node, trachea, and thymus cells (SEQ ID NOS:263-270).

Mitochondrial proteins that are encoded in the nucleus, including, but not limited to, UCPs, exert biological effect by regulating the efficiency of energy generation in the body, with the result being that excess resources are converted to heat or are otherwise stored as fat, etc. Regulating the function of such nuclear-encoded mitochondrial proteins will effect processes mediated by such proteins, with resulting effects on fat production and usage, superoxide generation and regulation, and all biological properties and functions that are tied to fatty acid metabolism. Because of these important roles, mitochondrial proteins that are encoded in the nucleus, including, but not limited to, UCPs, have been the focus of intense scientific scrutiny (see, e.g., PCT Patent Application Serial No. PCT/EP98/02645, and U.S. Pat. Nos. 5,853,975, 5,741,666 and 5,702,902, which describe a variety of uses, assays, and applications that can be applied to the presently described mitochondrial proteins).

Because of the diverse activities that have been associated with Notch signaling pathways, Notch receptors, and their associated ligands and antagonists, have been subject to intense scientific scrutiny (see, e.g., U.S. Pat. Nos. 5,786,158, 5,780,300, and 5,856,441). These patents provide examples of how the described Notch receptors and ligands can be produced, antagonized, used, processed, applied, and delivered. Given their structural relatedness, the described Notch ligand-like proteins are suitable for use and modification as contemplated for other Notch ligands and antagonists.

Given their structural relatedness, and the similarity of the four transmembrane regions, the described CD-like proteins are suitable for uses and applications previously described for similar proteins (see, e.g., U.S. Pat. Nos. 5,977,072 and 5,863,735).

Because of their medical relevance, GABA receptors have been subject to considerable scientific scrutiny, as evidenced by U.S. Pat. No. 6,043,054 (corresponding to PCT Patent Application No. PCT/US99/02904, Publication No. WO 99/42580 A2), which describes a variety of uses, assays, and applications that can be applied to the presently described GABA receptor subunit proteins.

Because of their important role in mediating kidney development, proteins that are similar to those described in SEQ ID NOS:104-107 (i.e., nephrins) have been the focus of intense scientific scrutiny (see, for example, PCT Patent Application Serial No. PCT/US99/05578, Publication Number WO 99/47562, and corresponding U.S. Pat. No. 6,207,811, which describe applications and uses that are applicable to the presently described proteins). Additionally, proteins related to those described in SEQ ID NOS:104-107 can serve as receptors for SLIT protein ligands (see, e.g., U.S. patent application Ser. No. 07/624,135, filed Dec. 7, 1990). Accordingly, an additional aspect of the present invention includes the use of slit proteins and slit protein homologs (as well as soluble versions of SEQ ID NOS:104-107) to treat kidney or adipose related diseases or disorders.

Given their structural relatedness of the presently described cadherin-like sequences to a variety of animal cadherin family proteins and the Drosophila tumor suppressor FAT, uses and applications relevant to the presently described cadherin-like sequences are described in U.S. Pat. No. 5,639,634.

The present invention encompasses the nucleotides presented in the Sequence Listing, host cells expressing such nucleotides, the expression products of such nucleotides, and: (a) nucleotides that encode mammalian homologs of the described nucleotides, including the specifically described human membrane proteins, and the human membrane protein products; (b) nucleotides that encode one or more portions of the human membrane proteins that correspond to functional domains, and the polypeptide products specified by such nucleotide sequences, including, but not limited to, the novel regions of any active domain(s); (c) isolated nucleotides that encode mutant versions, engineered or naturally occurring, of the described membrane proteins, in which all or a part of at least one domain is deleted or altered, and the polypeptide products specified by such nucleotide sequences, including, but not limited to, soluble proteins and peptides in which all or a portion of the signal sequence is deleted; (d) nucleotides that encode chimeric fusion proteins containing all or a portion of a coding region of a membrane protein, or one of its domains (e.g., a receptor or ligand binding domain, accessory protein/self-association domain, etc.) fused to another peptide or polypeptide; or (e) therapeutic or diagnostic derivatives of the described polynucleotides, such as oligonucleotides, antisense polynucleotides, ribozymes, dsRNA, or gene therapy constructs, comprising a sequence first disclosed in the Sequence Listing.

As discussed above, the present invention includes the human DNA sequences presented in the Sequence Listing (and vectors comprising the same), and additionally contemplates any nucleotide sequence encoding a contiguous membrane protein open reading frame (ORF) that hybridizes to a complement of a DNA sequence presented in the Sequence Listing under highly stringent conditions, e.g., hybridization to filter-bound DNA in 0.5 M NaHPO₄, 7% sodium dodecyl sulfate (SDS), 1 mM EDTA at 65° C., and washing in 0.1×SSC/0.1% SDS at 68° C. (“Current Protocols in Molecular Biology”, Vol. 1, p. 2.10.3 (Ausubel et al., eds., Green Publishing Associates, Inc., and John Wiley & Sons, Inc., New York, 1989)) and encodes a functionally equivalent expression product. Additionally contemplated are any nucleotide sequences that hybridize to the complement of a DNA sequence that encodes and expresses an amino acid sequence presented in the Sequence Listing under moderately stringent conditions, e.g., washing in 0.2×SSC/0.1% SDS at 42° C. (“Current Protocols in Molecular Biology”, supra), yet still encodes a functionally equivalent membrane protein product. Functional equivalents of a membrane protein include naturally occurring membrane proteins present in other species, and mutant membrane proteins, whether naturally occurring or engineered (by site directed mutagenesis, gene shuffling, directed evolution as described in, for example, U.S. Pat. No. 5,837,458). The invention also includes degenerate nucleic acid variants of the disclosed membrane protein polynucleotide sequences.

Additionally contemplated are polynucleotides encoding membrane protein ORFs, or their functional equivalents, encoded by polynucleotide sequences that are about 99, 95, 90, or about 85 percent similar or identical to corresponding regions of the nucleotide sequences of the Sequence Listing (as measured by BLAST sequence comparison analysis using, for example, the GCG sequence analysis package (the University of Wisconsin GCG sequence analysis package, SEQUENCHER 3.0, Gene Codes Corp., Ann Arbor, Mich.) using default settings).

The invention also includes nucleic acid molecules, preferably DNA molecules, that hybridize to, and are therefore the complements of, the described membrane protein nucleotide sequences. Such hybridization conditions may be highly stringent or less highly stringent, as described herein. In instances where the nucleic acid molecules are deoxyoligonucleotides, such molecules are generally about 16 to about 100 bases long, or about 20 to about 80 bases long, or about 34 to about 45 bases long, or any variation or combination of sizes represented therein that incorporate a contiguous region of sequence first disclosed in the Sequence Listing. Such oligonucleotides can be used in conjunction with the polymerase chain reaction (PCR) to screen libraries, isolate clones, and prepare cloning and sequencing templates, etc.

Alternatively, such membrane protein oligonucleotides can be used as hybridization probes for screening libraries, and assessing gene expression patterns (particularly using a microarray or high-throughput “chip” format). Additionally, a series of oligonucleotide sequences, or the complements thereof, can be used to represent all or a portion of the described membrane protein sequences. An oligonucleotide or polynucleotide sequence first disclosed in at least a portion of one or more of the sequences of SEQ ID NOS:1-270 can be used as a hybridization probe in conjunction with a solid support matrix/substrate (resins, beads, membranes, plastics, polymers, metal or metallized substrates, crystalline or polycrystalline substrates, etc.). Of particular note are spatially addressable arrays (i.e., gene chips, microtiter plates, etc.) of oligonucleotides and polynucleotides, or corresponding oligopeptides and polypeptides, wherein at least one of the biopolymers present on the spatially addressable array comprises an oligonucleotide or polynucleotide sequence first disclosed in at least one of the sequences of SEQ ID NOS:1-270, or an amino acid sequence encoded thereby. Methods for attaching biopolymers to, or synthesizing biopolymers on, solid support matrices, and conducting binding studies thereon, are disclosed in, inter alia, U.S. Pat. Nos. 5,700,637, 5,556,752, 5,744,305, 4,631,211, 5,445,934, 5,252,743, 4,713,326, 5,424,186, and 4,689,405.

Addressable arrays comprising sequences first disclosed in SEQ ID NOS:1-270 can be used to identify and characterize the temporal and tissue specific expression of a gene. These addressable arrays incorporate oligonucleotide sequences of sufficient length to confer the required specificity, yet be within the limitations of the production technology. The length of these probes is usually within a range of between about 8 to about 2000 nucleotides. Preferably the probes consist of 60 nucleotides, and more preferably 25 nucleotides, from the sequences first disclosed in SEQ ID NOS:1-270.

For example, a series of oligonucleotide sequences, or the complements thereof, can be used in chip format to represent all or a portion of the described membrane protein sequences. The oligonucleotides, typically between about 16 to about 40 (or any whole number within the stated range) nucleotides in length, can partially overlap each other, and/or the sequence may be represented using oligonucleotides that do not overlap. Accordingly, the described polynucleotide sequences shall typically comprise at least about two or three distinct oligonucleotide sequences of at least about 8 nucleotides in length that are each first disclosed in the described Sequence Listing. Such oligonucleotide sequences can begin at any nucleotide present within a sequence in the Sequence Listing, and proceed in either a sense (5′-to-3′) orientation vis-a-vis the described sequence or in an antisense orientation.

Microarray-based analysis allows the discovery of broad patterns of genetic activity, providing new understanding of gene functions, and generating novel and unexpected insight into transcriptional processes and biological mechanisms. The use of addressable arrays comprising sequences first disclosed in SEQ ID NOS:1-270 provides detailed information about transcriptional changes involved in a specific pathway, potentially leading to the identification of novel components, or gene functions that manifest themselves as novel phenotypes.

Probes consisting of sequences first disclosed in SEQ ID NOS:1-270 can also be used in the identification, selection, and validation of novel molecular targets for drug discovery. The use of these unique sequences permits the direct confirmation of drug targets, and recognition of drug dependent changes in gene expression that are modulated through pathways distinct from the intended target of the drug. These unique sequences therefore also have utility in defining and monitoring both drug action and toxicity.

As an example of utility, the sequences first disclosed in SEQ ID NOS:1-270 can be utilized in microarrays, or other assay formats, to screen collections of genetic material from patients who have a particular medical condition. These investigations can also be carried out using the sequences first disclosed in SEQ ID NOS:1-270 in silico, and by comparing previously collected genetic databases and the disclosed sequences using computer software known to those in the art.

Thus the sequences first disclosed in SEQ ID NOS:1-270 can be used to identify mutations associated with a particular disease, and also in diagnostic or prognostic assays.

Although the presently described sequences have been specifically described using nucleotide sequence, it should be appreciated that each of the sequences can uniquely be described using any of a wide variety of additional structural attributes, or combinations thereof. For example, a given sequence can be described by the net composition of the nucleotides present within a given region of the sequence, in conjunction with the presence of one or more specific oligonucleotide sequence(s) first disclosed in SEQ ID NOS:1-270. Alternatively, a restriction map specifying the relative positions of restriction endonuclease digestion sites, or various palindromic or other specific oligonucleotide sequences, can be used to structurally describe a given sequence. Such restriction maps, which are typically generated by widely available computer programs (e.g., the University of Wisconsin GCG sequence analysis package, SEQUENCHER 3.0, Gene Codes Corp., etc.), can optionally be used in conjunction with one or more discrete nucleotide sequence(s) present in the sequence that can be described by the relative position of the sequence relative to one or more additional sequence(s) or one or more restriction sites present in the disclosed sequence.

For oligonucleotide probes, highly stringent conditions may refer, e.g., to washing in 6×SSC/0.05% sodium pyrophosphate at 37° C. (for 14-base oligos), 48° C. (for 17-base oligos), 55° C. (for 20-base oligos), and 60° C. (for 23-base oligos). These nucleic acid molecules may encode or act as antisense molecules, useful, for example, in membrane protein gene regulation and/or as antisense primers in amplification reactions of membrane protein nucleic acid sequences. With respect to membrane protein gene regulation, such techniques can be used to regulate biological functions. Further, such sequences may be used as part of ribozyme and/or triple helix sequences that are also useful for membrane protein gene regulation.

Inhibitory antisense or double stranded oligonucleotides can additionally comprise at least one modified base moiety that is selected from the group including, but not limited to, 5-fluorouracil, 5-bromouracil, 5-chlorouracil, 5-iodouracil, hypoxanthine, xanthine, 5-(carboxyhydroxylmethyl) uracil, 4-acetylcytosine, 5-carboxymethylaminomethyl-2-thiouridine, 5-carboxymethylaminomethyluracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, queosine, 5-methoxyuracil, 2-methylthio-N6-isopentenyladenine, pseudouracil, uracil-5-oxyacetic acid (v), wybutoxosine, 2-thiocytosine, 5-methyl-2-thiouracil, 2-thiouracil, 4-thiouracil, 5-methyluracil, dihydrouracil, uracil-5-oxyacetic acid methylester, (acp3)w, 3-(3-amino-3-N-2-carboxypropyl) uracil, and 2,6-diaminopurine.

The antisense oligonucleotide can also comprise at least one modified sugar moiety selected from the group including, but not limited to, arabinose, 2-fluoroarabinose, xylulose, and hexose.

In yet another embodiment, the antisense oligonucleotide will comprise at least one modified phosphate backbone selected from the group including, but not limited to, a phosphorothioate, a phosphorodithioate, a phosphoramidothioate, a phosphoramidate, a phosphordiamidate, a methylphosphonate, an alkyl phosphotriester, and a formacetal or analog thereof.

In yet another embodiment, the antisense oligonucleotide is an α-anomeric oligonucleotide. An α-anomeric oligonucleotide forms specific double-stranded hybrids with complementary RNA in which, contrary to the usual β-units, the strands run parallel to each other (Gautier et al., Nucl. Acids Res. 15:6625-6641, 1987). The oligonucleotide is a 2′-0-methylribonucleotide (Inoue et al., Nucl. Acids Res. 15:6131-6148, 1987), or a chimeric RNA-DNA analogue (Inoue et al., FEBS Lett. 215:327-330, 1987). Alternatively, double stranded RNA can be used to disrupt the expression and function of a targeted membrane protein.

Oligonucleotides of the invention can be synthesized by standard methods known in the art, e.g., by use of an automated DNA synthesizer (such as are commercially available from Biosearch, Applied Biosystems, etc.). As examples, phosphorothioate oligonucleotides can be synthesized (Stein et al., Nucl. Acids Res. 16:3209-3221, 1988), and methylphosphonate oligonucleotides can be prepared by use of controlled pore glass polymer supports (Sarin et al., Proc. Natl. Acad. Sci. USA 85:7448-7451, 1988), etc.

Low stringency conditions are well-known to those of skill in the art, and will vary predictably depending on the specific organisms from which the library and the labeled sequences are derived. For guidance regarding such conditions, see, for example, “Molecular Cloning, A Laboratory Manual” (Sambrook et al., eds., Cold Spring Harbor Press, Cold Spring Harbor, N.Y., 1989), “Current Protocols in Molecular Biology”, supra, and periodic updates thereof.

Alternatively, suitably labeled membrane protein nucleotide probes can be used to screen a human genomic library using appropriately stringent conditions or by PCR. The identification and characterization of human genomic clones is helpful for identifying polymorphisms (including, but not limited to, nucleotide repeats, microsatellite alleles, single nucleotide polymorphisms, or coding single nucleotide polymorphisms), determining the genomic structure of a given locus/allele, and designing diagnostic tests. For example, sequences derived from regions adjacent to the intron/exon boundaries of the human gene can be used to design primers for use in amplification assays to detect mutations within the exons, introns, splice sites (e.g., splice acceptor and/or donor sites), etc., that can be used in diagnostics and pharmacogenomics.

For example, the present sequences can be used in restriction fragment length polymorphism (RFLP) analysis to identify specific individuals. In this technique, an individual's genomic DNA is digested with one or more restriction enzymes, and probed on a Southern blot to yield unique bands for identification (as generally described in U.S. Pat. No. 5,272,057). In addition, the sequences of the present invention can be used to provide polynucleotide reagents, e.g., PCR primers, targeted to specific loci in the human genome, which can enhance the reliability of DNA-based forensic identifications by, for example, providing another “identification marker” (i.e., another DNA sequence that is unique to a particular individual). Actual base sequence information can be used for identification as an accurate alternative to patterns formed by restriction enzyme generated fragments.

Further, a membrane protein gene homolog can be isolated from nucleic acid from an organism of interest by performing PCR using two degenerate or “wobble” oligonucleotide primer pools designed on the basis of amino acid sequences within the membrane protein products disclosed herein. The template for the reaction may be genomic DNA, or total RNA, mRNA, and/or cDNA obtained by reverse transcription of mRNA prepared from human or non-human cell lines or tissue known to express, or suspected of expressing, an allele of a membrane protein gene. The PCR product can be subcloned and sequenced to ensure that the amplified sequences represent the sequence of the desired membrane protein gene. The PCR fragment can then be used to isolate a full length cDNA clone by a variety of methods. For example, the amplified fragment can be labeled and used to screen a cDNA library, such as a bacteriophage cDNA library. Alternatively, the labeled fragment can be used to isolate genomic clones via the screening of a genomic library.

PCR technology can also be used to isolate full length cDNA sequences. For example, RNA can be isolated, following standard procedures, from an appropriate cellular or tissue source (i.e., one known to express, or suspected of expressing, a membrane protein gene). A reverse transcription (RT) reaction can be performed on the RNA using an oligonucleotide primer specific for the most 5′ end of the amplified fragment for the priming of first strand synthesis. The resulting RNA/DNA hybrid may then be “tailed” using a standard terminal transferase reaction, the hybrid may be digested with RNase H, and second strand synthesis may then be primed with a complementary primer. Thus, cDNA sequences upstream of the amplified fragment can be isolated. For a review of cloning strategies that can be used, see, e.g., “Molecular Cloning, A Laboratory Manual”, supra.

A cDNA encoding a mutant membrane protein sequence can be isolated, for example, by using PCR. In this case, the first cDNA strand may be synthesized by hybridizing an oligo-dT oligonucleotide to mRNA isolated from tissue known to express, or suspected of expressing, a membrane protein, in an individual putatively carrying a mutant membrane protein allele, and by extending the new strand with reverse transcriptase. The second strand of the cDNA is then synthesized using an oligonucleotide that hybridizes specifically to the 5′ end of the normal sequence. Using these two primers, the product is then amplified via PCR, optionally cloned into a suitable vector, and subjected to DNA sequence analysis through methods well-known to those of skill in the art. By comparing the DNA sequence of the mutant membrane protein allele to that of a corresponding normal membrane protein allele, the mutation(s) responsible for the loss or alteration of function of the mutant membrane protein gene product can be ascertained.

Alternatively, a genomic library can be constructed using DNA obtained from an individual suspected of carrying, or known to carry, a mutant membrane protein allele (e.g., a person manifesting a membrane protein-associated phenotype such as, for example, arthritis, osteoporosis, obesity, high blood pressure, connective tissue disorders, paralysis or palsy, nerve damage or degeneration, inflammatory disorders, vision disorders, depression, seizures, infertility, cancer, etc.), or a cDNA library can be constructed using RNA from a tissue known to express, or suspected of expressing, a mutant membrane protein allele. A normal membrane protein gene, or any suitable fragment thereof, can then be labeled and used as a probe to identify the corresponding mutant membrane protein allele in such libraries. Clones containing mutant membrane protein sequences can then be purified and subjected to sequence analysis according to methods well-known to those skilled in the art.

Additionally, an expression library can be constructed utilizing cDNA synthesized from, for example, RNA isolated from a tissue known to express, or suspected of expressing, a mutant membrane protein allele in an individual suspected of carrying, or known to carry, such a mutant allele. In this manner, gene products made by the putatively mutant tissue can be expressed and screened using standard antibody screening techniques in conjunction with antibodies raised against a normal membrane protein product, as described below (for screening techniques, see, for example, “Antibodies: A Laboratory Manual” (Harlow and Lane, eds., Cold Spring Harbor Press, Cold Spring Harbor, N.Y., 1988)).

Additionally, screening can be accomplished by screening with labeled membrane protein fusion proteins, such as, for example, alkaline phosphatase-membrane protein or membrane protein-alkaline phosphatase fusion proteins. In cases where a membrane protein mutation results in an expression product with altered function (e.g., as a result of a missense or a frameshift mutation), polyclonal antibodies to a membrane protein are likely to cross-react with a corresponding mutant membrane protein expression product. Library clones detected via their reaction with such labeled antibodies can be purified and subjected to sequence analysis according to methods well-known in the art.

The invention also encompasses: (a) DNA vectors that contain any of the foregoing membrane protein coding sequences and/or their complements (i.e., antisense); (b) DNA expression vectors that contain any of the foregoing membrane protein coding sequences operatively associated with a regulatory element that directs the expression of the coding sequences (for example, baculovirus as described in U.S. Pat. No. 5,869,336); (c) genetically engineered host cells that contain any of the foregoing membrane protein coding sequences operatively associated with a regulatory element that directs the expression of the coding sequences in the host cell; and (d) genetically engineered host cells that express an endogenous membrane protein sequence under the control of an exogenously introduced regulatory element (i.e., gene activation). As used herein, regulatory elements include, but are not limited to, inducible and non-inducible promoters, enhancers, operators, and other elements known to those skilled in the art that drive and regulate expression. Such regulatory elements include, but are not limited to, the cytomegalovirus (hCMV) immediate early gene, regulatable, viral elements (particularly retroviral LTR promoters), the early or late promoters of SV40 or adenovirus, the lac system, the trp system, the TAC system, the TRC system, the major operator and promoter regions of phage lambda, the control regions of fd coat protein, the promoter for 3-phosphoglycerate kinase (PGK), the promoters of acid phosphatase, and the promoters of the yeast α-mating factors.

The present invention also encompasses antibodies and anti-idiotypic antibodies (including Fab fragments), antagonists and agonists of the described membrane proteins, as well as compounds or nucleotide constructs that inhibit expression of a membrane protein sequence (transcription factor inhibitors, antisense and ribozyme molecules, or open reading frame sequence or regulatory sequence replacement constructs), or promote the expression of a membrane protein (e.g., expression constructs in which membrane protein coding sequences are operatively associated with expression control elements such as promoters, promoter/enhancers, etc.).

The membrane proteins, peptides, fusion proteins, nucleotide sequences, antibodies, antagonists, and agonists can be useful for the detection of mutant membrane proteins, or inappropriately expressed membrane proteins, for the diagnosis of disease. The membrane proteins, peptides, fusion proteins, nucleotide sequences, host cell expression systems, antibodies, antagonists, agonists, and genetically engineered cells and animals can be used for screening for drugs (or high throughput screening of combinatorial libraries) effective in the treatment of the symptomatic or phenotypic manifestations of perturbing the normal function of a membrane protein in the body. The use of engineered host cells and/or animals may offer an advantage in that such systems allow not only for the identification of compounds that bind to the endogenous receptor for a membrane protein, but can also identify compounds that trigger membrane protein-mediated activities or pathways.

Finally, the membrane protein products can be used as therapeutics. For example, soluble derivatives, such as peptides/domains corresponding to membrane proteins, membrane protein fusion protein products (especially membrane protein-Ig fusion proteins, i.e., fusions of a membrane protein, or a domain of a membrane protein, to an IgFc), membrane protein antibodies and anti-idiotypic antibodies (including Fab fragments), antagonists or agonists (including compounds that modulate or act on downstream targets in a membrane protein-mediated pathway), can be used to directly treat diseases, including infectious diseases, or disorders. For instance, the administration of an effective amount of a soluble membrane protein, a membrane protein-IgFc fusion protein, or an anti-idiotypic antibody (or its Fab) that mimics a membrane protein, could activate or effectively antagonize an endogenous membrane protein receptor. Nucleotide constructs encoding such membrane protein products can be used to genetically engineer host cells to express such products in vivo; these genetically engineered cells function as “bioreactors” in the body delivering a continuous supply of a membrane protein, peptide, or fusion protein to the body. Nucleotide constructs encoding functional membrane proteins, mutant membrane proteins, as well as antisense and ribozyme molecules, can also be used in “gene therapy” approaches for the modulation of membrane protein expression. Thus, the invention also encompasses pharmaceutical formulations and methods for treating biological disorders.

Various aspects of the invention are described in greater detail in the subsections below.

### 7.1 Membrane Protein Nucleic Acid Sequences

The cDNA sequences and the corresponding deduced amino acid sequences of the described membrane proteins are presented in the Sequence Listing. The membrane protein nucleotide sequences were obtained from: human lymph node, kidney, and fetal brain cDNA libraries (SEQ ID NOS:1-4); clustered human gene trapped sequences, ESTs and human kidney and bone marrow cDNA libraries (SEQ ID NOS:5-8); human gene trapped sequence tags and cDNA clones from a human testis cDNA library (SEQ ID NOS:9-22); clustered human gene trapped sequences, and ESTs from human testis cells (SEQ ID NOS:23-31); clustered human gene trapped sequences, and clones from human trachea, pituitary, and lung cDNA libraries (SEQ ID NOS:32-35); clustered human gene trapped sequences, ESTs, and cDNAs isolated from human brain, fetal brain, cerebellum, and hypothalamus cDNA libraries (SEQ ID NOS:36-62); clustered human gene trapped sequences, ESTs, and cDNA isolated from a human testis cell library (SEQ ID NOS:63-74); clustered human gene trapped sequences, genomic sequence, ESTs, cDNAs from a human brain cDNA library, and human RT-PCR cDNA products from RNA preparations from human brain, cerebellum, hypothalamus, pituitary, and spinal cord (SEQ ID NOS:75-91); a human pituitary gland cDNA library, and RT-PCR products generated using fetal brain, brain, pituitary gland, and testis mRNA (SEQ ID NOS:92-103); a human kidney cDNA library and RACE products generated using human adipose and fetal kidney mRNA (SEQ ID NOS:104-107); clustered human sequences, cDNA isolated from a human brain cDNA library, as well as cDNAs made from fetal brain, adult brain, pituitary gland, and cerebellum mRNA (SEQ ID NOS:108-112); clustered human gene trapped sequences, genomic sequence, ESTs, and cDNAs from a skeletal muscle, bone marrow, and lymph node cDNA libraries (SEQ ID NOS:113-117); clustered genomic sequence, ESTs, and cDNAs from a trachea cDNA library (SEQ ID NOS:118-124); clustered genomic sequence encoded on human chromosome 8 (see GenBank Accession Number AC012215), ESTs, and cDNAs from testis, prostate, adrenal gland, kidney, and pituitary mRNAs (SEQ ID NOS:125-157); aligning cDNAs from bone marrow, lymph node, kidney, thymus, trachea, fetal kidney, fetal brain, testis, kidney, placenta, thymus, pituitary, and fetal mRNAs and human genomic DNA sequence (SEQ ID NOS:158-192); clustered human ESTs, and cDNAs made from brain mRNA (SEQ ID NOS:193-200); clustered genomic sequence, ESTs, and cDNAs from testis mRNA (SEQ ID NOS:201-203); clustered genomic sequence, ESTs, and cDNAs from skeletal muscle, trachea, lung, fetal kidney, fetal lung, lymph node, fetus, and bone marrow mRNAs (SEQ ID NOS:204 and 205); clustered genomic sequence, ESTs, and cDNAs produced using activated lymph node, fetal kidney, and fetus mRNAs (SEQ ID NOS:206-214); clustered genomic sequence, ESTs, and cDNAs produced using lymph node, bone marrow, and testis mRNAs (SEQ ID NOS:215-218); cDNAs prepared and isolated from human spinal cord, fetal brain, 6- and 9-week old embryos, cerebellum, lymph node and testis mRNAs (SEQ ID NOS:219-221); clustered human genomic sequences, ESTs, and human cDNAs made from testis, fetal kidney, kidney, mammary gland, and placenta mRNAs (SEQ ID NOS:222-228); clustered genomic sequence, ESTs, and cDNAs produced using human spleen, lymph node, and testis mRNAs (SEQ ID NOS:229-233); clustered genomic sequence, ESTs, and cDNAs produced using testis and kidney mRNAs (SEQ ID NOS:234-236); clustered genomic sequence, ESTs, and cDNAs produced using fetal brain, testis, fetal lung, lymph node, and fetal kidney mRNAs (SEQ ID NOS:237-240); clustered human sequences and cDNAs made from human lung, fetal brain, and adult brain mRNAs (SEQ ID NOS:241-262); and clustered genomic sequence, ESTs, and cDNAs produced using human thyroid and trachea mRNAs (SEQ ID NOS:263-270). The mRNAs and cDNA libraries were purchased from Clontech (Palo Alto, Calif.) and/or Edge Biosystems (Gaithersburg, Md.).

The genes encoding the described membrane proteins are apparently encoded on: human chromosome 1 (SEQ ID NOS:104-107); human chromosome 5, see GenBank Accession Number AC010457 (SEQ ID NOS:113-117); human chromosome 6, see GenBank Accession Number AL354719 (SEQ ID NOS:118-124); human chromosome 8, see GenBank Accession Number AC012215 (SEQ ID NOS:125-157); human chromosome 15, see GenBank Accession Number AC058820 (SEQ ID NOS:158-192); human chromosome 11, see GenBank Accession Number AC024231 (SEQ ID NOS:193-200); a single coding exon present on human chromosome 10, see GenBank Accession Number AL022345 (SEQ ID NOS:201-203); human chromosome 3, see GenBank Accession Number AC024888 (SEQ ID NOS:204 and 205); human chromosome 1, see GenBank Accession Number AC027070 (SEQ ID NOS:206-214); human chromosome 12, see GenBank Accession Number AC006927 (SEQ ID NOS:215-218); human chromosome 1, see GenBank Accession Number AC015973 (SEQ ID NOS:219-221); human chromosome 7, see GenBank Accession Number AC073468 (SEQ ID NOS:222-228); human chromosome 12, see GenBank Accession Number AC012555 (SEQ ID NOS:229-233); human chromosome 3, see GenBank Accession Number AC069529 (SEQ ID NOS:234-236); human chromosome 9, see GenBank Accession Number AL160274 (SEQ ID NOS:237-240); human chromosome 7, see GenBank Accession Number AC004836 (SEQ ID NOS:241-262); and human chromosome 8, see GenBank Accession Number AC021001 (SEQ ID NOS:263-270). As such, the described sequences can also be used to map the corresponding coding regions of the human genome, and to verify/identify mRNA exon splice junctions.

Several polymorphisms were identified, including: an A/G polymorphism at nucleotide (“nt”) position 1108 of SEQ ID NOS:5 and 7 (denoted by an “r” in the Sequence Listing), which can result in an isoleucine or valine residue at corresponding amino acid (“aa”) position 370 of SEQ ID NOS:6 and 8; a G/A polymorphism at nt position 1177 of SEQ ID NOS:9, 11, 13, 15, 17, 19, and 21 (denoted by an “r” in the Sequence Listing), which can result in a glutamate or lysine residue at corresponding aa position 393 of SEQ ID NOS:10, 12, 14, 16, 18, 20, and 22; a C/T polymorphism at nt position 812 of SEQ ID NOS:36, 38, 46, 48, 50, 52, 54, and 56 (denoted by a “y” in the Sequence Listing), which can result in a serine or leucine residue at corresponding aa position 271 of SEQ ID NOS:37, 39, 47, 49, 51, 53, 55, and 57; a G/A polymorphism at nt position 1024 of SEQ ID NOS:36, 46, 50, and 54 (denoted by an “r” in the Sequence Listing); which can result in an aspartate or asparagine residue at corresponding aa position 342 of SEQ ID NOS:37, 47, 51, and 55; a transcriptionally silent (“silent”) T/C polymorphism at nt position 1116 of SEQ ID NOS:36, 46, 50, and 54, and nt position 972 of SEQ ID NOS:38, 48, 52, and 56 (denoted by a “y” in the Sequence Listing), both of which result in a valine residue at corresponding aa position 372 of SEQ ID NOS:37, 47, 51, and 55, and aa position 324 of SEQ ID NOS:39, 49, 53, and 57; a C/T polymorphism at nt position 808 of SEQ ID NO:44, which can result in an arginine or tryptophan residue at corresponding aa position 270 of SEQ ID NO:45; a silent C/T polymorphism at nt position 99 of SEQ ID NOS:75, 77, 79, and 81, both of which result in an aspartate residue at corresponding aa position 33 of SEQ ID NOS:76, 78, 80, and 82; a C/A polymorphism at nt position 758 of SEQ ID NOS:75, 77, 79, and 81, and nt position 356 of SEQ ID NOS:83, 85, 87, and 89 (denoted by an “m” in the Sequence Listing), which can result in a serine or tyrosine residue at corresponding aa position 253 of SEQ ID NOS:76, 78, 80, and 82, and aa position 119 of SEQ ID NOS:84, 86, 88, and 90; a silent G/C polymorphism at nt position 750 of SEQ ID NO:104, and nt position 144 of SEQ ID NO:106 (denoted by an “s” in the Sequence Listing), both of which result in a proline residue at corresponding aa position 250 of SEQ ID NO:105, and aa position 48 of SEQ ID NO:107; a silent G/A polymorphism at nt position 264 of SEQ ID NO:108 (denoted by an “r” in the Sequence Listing), both of which result in a threonine residue at corresponding aa position 88 of SEQ ID NO:109; a silent A/T polymorphism at nt position 762 of SEQ ID NO:108 (denoted by a “w” in the Sequence Listing), both of which result in a serine residue at corresponding aa position 254 of SEQ ID NO:109; a silent G/T polymorphism at nt position 771 of SEQ ID NO:108 (denoted by a “k” in the Sequence Listing), both of which result in a tyrosine residue at corresponding aa position 257 of SEQ ID NO:109; a G/T polymorphism at nt position 1161 of SEQ ID NO:113 (denoted by a “k” in the Sequence Listing), which can result in a glutamine or histidine residue at corresponding aa position 387 of SEQ ID NO:114; a G/C polymorphism at nt position 2530 of SEQ ID NO:113 (denoted by an “s” in the Sequence Listing), which can result in an aspartate or histidine residue at corresponding aa position 844 of SEQ ID NO:114; an A/C polymorphism at nt position 661 of SEQ ID NO:118, and nt position 208 of SEQ ID NO:122 (denoted by an “m” in the Sequence Listing), which can result in a threonine or proline residue at corresponding aa position 221 of SEQ ID NO:119, and aa position 70 of SEQ ID NO:123; a silent A/C polymorphism at nt position 330 of SEQ ID NO:120 (denoted by an “m” in the Sequence Listing), both of which result in a valine residue at corresponding aa position 110 of SEQ ID NO:121; a G/C polymorphism at nt position 776 of SEQ ID NOS:125, 127, 129, 131, 133, 135, 137, and 139, and nt position 83 of SEQ ID NOS:141, 143, 145, 147, 149, 151, 153, and 155, which can result in a serine or threonine residue at corresponding aa position 259 of SEQ ID NOS:126, 128, 130, 132, 134, 136, 138, and 140, and aa position 28 of SEQ ID NOS:142, 144, 146, 148, 150, 152, 154, and 156, respectively; a T/C polymorphism at nt position 788 of SEQ ID NOS:125, 127, 129, 131, 133, 135, 137, and 139, and nt position 95 of SEQ ID NOS:141, 143, 145, 147, 149, 151, 153, and 155, which can result in a valine or alanine residue at corresponding aa position 263 of SEQ ID NOS:126, 128, 130, 132, 134, 136, 138, and 140, and aa position 32 of SEQ ID NOS:142, 144, 146, 148, 150, 152, 154, and 156, respectively; a C/T polymorphism at nt position 1276 of SEQ ID NOS:125 and 133, nt position 1243 of SEQ ID NOS:127 and 135, nt position 1234 of SEQ ID NOS:129 and 137, nt position 1201 of SEQ ID NOS:131 and 139, nt position 583 of SEQ ID NOS:141 and 149, nt position 550 of SEQ ID NOS:143 and 151, nt position 541 of SEQ ID NOS:145 and 153, and nt position 508 of SEQ ID NOS:147 and 155, which can result in a leucine or phenylalanine residue at corresponding aa position 426 of SEQ ID NOS:126 and 134, aa position 415 of SEQ ID NOS:128 and 136, aa position 412 of SEQ ID NOS:130 and 138, aa position 401 of SEQ ID NOS:132 and 140, aa position 195 of SEQ ID NOS:142 and 150, aa position 184 of SEQ ID NOS:144 and 152, aa position 181 of SEQ ID NOS:146 and 154, and aa position 170 of SEQ ID NOS:148 and 156, respectively; a silent C/T polymorphism at nt position 1425 of SEQ ID NOS:158, 160, 162, 164, 166, 168, 170, 172, and 174, and nt position 1266 of SEQ ID NOS:176, 178, 180, 182, 184, 186, 188, and 190, both of which result in a phenylalanine residue at corresponding aa position 475 of SEQ ID NOS:159, 161, 163, 165, 167, 169, 171, 173, and 175, and aa position 422 of SEQ ID NOS:177, 179, 181, 183, 185, 187, 189, and 191; a silent G/A polymorphism at nt position 2502 of SEQ ID NOS:168 and 172, nt position 3387 of SEQ ID NOS:170 and 174, nt position 2343 of SEQ ID NO:186, and nt position 3228 of SEQ ID NOS:188 and 190, both of which result in a proline residue at corresponding aa position 834 of SEQ ID NOS:169 and 173, aa position 1129 of SEQ ID NOS:171 and 175, aa position 781 of SEQ ID NO:187, and aa position 1076 of SEQ ID NOS:189 and 191; an A/T polymorphism at nt position 4543 of SEQ ID NOS:193, 195, and 199, and nt position 4531 of SEQ ID NO:197, which can result in a threonine or serine residue at corresponding aa position 1515 of SEQ ID NOS:194, 196, and 200, and aa position 1511 of SEQ ID NO:198; an A/G polymorphism at nt position 4775 of SEQ ID NOS:193, 195, and 199, and nt position 4763 of SEQ ID NO:197, which can result in an aspartate or glycine residue at corresponding aa position 1592 of SEQ ID NOS:194, 196, and 200, and aa position 1588 of SEQ ID NO:198; an A/G polymorphism at nt position 6878 of SEQ ID NOS:193, 195, and 199, and nt position 6866 of SEQ ID NO:197, which can result in an asparagine or serine residue at corresponding aa position 2293 of SEQ ID NOS:194, 196, and 200, and aa position 2289 of SEQ ID NO:198; a G/C polymorphism at nt position 7227 of SEQ ID NOS:193, 195, and 199, and nt position 7215 of SEQ ID NO:197, which can result in an arginine or proline residue at corresponding aa position 2409 of SEQ ID NOS:194, 196, and 200, and aa position 2405 of SEQ ID NO:198; a G/A polymorphism at nt position 8263 of SEQ ID NOS:193, 195, and 199, and nt position 8251 of SEQ ID NO:197, which can result in a valine or isoleucine residue at corresponding aa position 2755 of SEQ ID NOS:194, 196, and 200, and aa position 2751 of SEQ ID NO:198; a G/A polymorphism at nt position 10552 of SEQ ID NOS:193, 195, and 199, and nt position 10540 of SEQ ID NO:197, which can result in valine or leucine residue at corresponding aa position 3518 of SEQ ID NOS:194, 196, and 200, and aa position 3514 of SEQ ID NO:198; a G/A polymorphism at nt position 11434 of SEQ ID NOS:193, 195, and 199, and nt position 11422 of SEQ ID NO:197, which can result in a glycine or serine residue at corresponding aa position 3812 of SEQ ID NOS:194, 196, and 200, and aa position 3808 of SEQ ID NO:198; a C/A polymorphism at nt position 12691 of SEQ ID NO:193, nt position 12679 of SEQ ID NO:197, and nt position 12688 of SEQ ID NO:199, which can result in a proline or threonine residue at corresponding aa position 4231 of SEQ ID NO:194, aa position 4227 of SEQ ID NO:198, and aa position 4230 of SEQ ID NO:200; a G/A polymorphism at nt position 12770 of SEQ ID NO:193, nt position 12758 of SEQ ID NO:197, and nt position 12767 of SEQ ID NO:199, which can result in a glycine or glutamate residue at corresponding aa position 4257 of SEQ ID NO:194, aa position 4253 of SEQ ID NO:198, and aa position 4256 of SEQ ID NO:200; a C/G polymorphism at nt position 12820 of SEQ ID NO:193, nt position 12808 of SEQ ID NO:197, and nt position 12817 of SEQ ID NO:199, which can result in a leucine or valine at corresponding aa position 4274 of SEQ ID NO:194, aa position 4270 of SEQ ID NO:198, and aa position 4273 of SEQ ID NO:200; a silent T/G polymorphism at nt position 336 of SEQ ID NOS:206 and 210, and nt position 444 of SEQ ID NOS:208 and 212, both of which result in an alanine residue at corresponding aa position 112 of SEQ ID NOS:207 and 211, and aa position 148 of SEQ ID NOS:209 and 213; a C/G polymorphism at nt position 424 of SEQ ID NOS:206 and 210, and nt position 532 of SEQ ID NOS:208 and 212, which can result in a glutamine or glutamate residue at corresponding aa position 142 of SEQ ID NOS:207 and 211, and aa position 178 of SEQ ID NOS:209 and 213; a silent C/G polymorphism at nt position 864 of SEQ ID NO:206, and nt position 972 of SEQ ID NO:208, both of which result in a proline residue at corresponding aa position 288 of SEQ ID NO:207, and aa position 324 of SEQ ID NO:209; a G/C polymorphism at nt position 901 of SEQ ID NO:206, and nt position 1009 of SEQ ID NO:208, which can result in an alanine or proline residue at corresponding aa position 301 of SEQ ID NO:207, and aa position 337 of SEQ ID NO:209; a T/C polymorphism at nt position 103 of SEQ ID NO:217 (denoted by a “y” in the Sequence Listing), which can result in a proline or serine residue at corresponding aa position 35 of SEQ ID NO:218; a silent T/C polymorphism at nt position 1644 of SEQ ID NO:229, and nt position 1737 of SEQ ID NO:231, both of which result in an asparagine residue at corresponding aa position 548 of SEQ ID NO:230, and aa position 579 of SEQ ID NO:232; a silent T/C polymorphism at nt position 2349 of SEQ ID NO:229, and nt position 2442 of SEQ ID NO:231, both of which result in a cysteine residue at corresponding aa position 783 of SEQ ID NO:230, and aa position 814 of SEQ ID NO:232; a silent T/C polymorphism at nt position 3162 of SEQ ID NO:229, and nt position 3255 of SEQ ID NO:231, both of which result in a histidine residue at corresponding aa position 1054 of SEQ ID NO:230, and aa position 1085 of SEQ ID NO:232; a silent C/T polymorphism at nt position 3405 of SEQ ID NO:229, and nt position 3498 of SEQ ID NO:231, both of which result in an asparagine residue at corresponding aa position 1135 of SEQ ID NO:230, and aa position 1166 of SEQ ID NO:232; an A/C polymorphism at nt position 4024 of SEQ ID NO:229, and nt position 4117 of SEQ ID NO:231, which can result in an asparagine or histidine residue at corresponding aa position 1342 of SEQ ID NO:230, and aa position 1373 of SEQ ID NO:232; a silent G/A polymorphism at nt position 4293 of SEQ ID NO:229, and nt position 4386 of SEQ ID NO:231, both of which result in an alanine residue at corresponding aa position 1431 of SEQ ID NO:230, and aa position 1462 of SEQ ID NO:232; a silent C/T polymorphism at nt position 4320 of SEQ ID NO:229, and nt position 4413 of SEQ ID NO:231, both of which result in a phenylalanine residue at corresponding aa position 1440 of SEQ ID NO:230, and aa position 1471 of SEQ ID NO:232; a C/G polymorphism at nt position 5506 of SEQ ID NO:229, and nt position 5599 of SEQ ID NO:231, which can result in a leucine or valine residue at corresponding aa position 1836 of SEQ ID NO:230, and aa position 1867 of SEQ ID NO:232; a GC/TG polymorphism at nt positions 5704 and 5705 of SEQ ID NO:229, and nt positions 5797 and 5798 of SEQ ID NO:231, which can result in an alanine or cysteine residue at corresponding aa position 1902 of SEQ ID NO:230, and aa position 1933 of SEQ ID NO:232; a T/A polymorphism at nt position 5780 of SEQ ID NO:229, and nt position 5873 of SEQ ID NO:231, which can result in a phenylalanine or tyrosine residue at corresponding aa position 1927 of SEQ ID NO:230, and aa position 1958 of SEQ ID NO:232; an A/C polymorphism at nt position 871 of SEQ ID NOS:241, 249, 251, and 261, nt position 778 of SEQ ID NOS:243, 253, 257, and 259, and nt position 55 of SEQ ID NOS:247 and 255, which can result in a threonine or serine residue at corresponding aa position 291 of SEQ ID NOS:242, 250, 252, and 262, aa position 260 of SEQ ID NOS:244, 254, 258, and 260, and aa position 19 of SEQ ID NOS:248 and 256; an A/G polymorphism at nt position 878 of SEQ ID NOS:241, 249, 251, and 261, nt position 785 of SEQ ID NOS:243, 253, 257, and 259, and nt position 62 of SEQ ID NOS:247 and 255, which can result in a glutamine or proline residue at corresponding aa position 293 of SEQ ID NOS:242, 250, 252, and 262, aa position 262 of SEQ ID NOS:244, 254, 258, and 260, and aa position 21 of SEQ ID NOS:248 and 256; a G/T polymorphism at nt position 1430 of SEQ ID NOS:241 and 251, nt position 1337 of SEQ ID NOS:243 and 259, nt position 614 of SEQ ID NO:247, nt position 1355 of SEQ ID NOS:249 and 261, nt position 1262 of SEQ ID NO:253, and nt position 539 of SEQ ID NO:255, which can result in an arginine or isoleucine residue at corresponding aa position 477 of SEQ ID NO:242 and 252, aa position 446 of SEQ ID NOS:244 and 260, aa position 205 of SEQ ID NO:248, aa position 452 of SEQ ID NOS:250 and 262, aa position 421 of SEQ ID NO:254, and aa position 180 of SEQ ID NO:256; a T/G polymorphism at nt position 2579 of SEQ ID NO:241, nt position 2486 of SEQ ID NO:243, nt position 1763 of SEQ ID NO:247, nt position 2504 of SEQ ID NO:249, nt position 2411 of SEQ ID NO:253, and nt position 1688 of SEQ ID NO:255, which can result in a valine or glycine residue at corresponding aa position 860 of SEQ ID NO:242, aa position 829 of SEQ ID NO:244, aa position 588 of SEQ ID NO:248, aa position 835 of SEQ ID NO:250, aa position 804 of SEQ ID NO:254, and aa position 563 of SEQ ID NO:256; a silent A/G polymorphism at nt position 159 of SEQ ID NOS:263, 265, 267, and 269, both of which result in a glycine residue at corresponding aa position 53 of SEQ ID NOS:264, 266, 268, and 270; a C/T polymorphism at nt position 472 of SEQ ID NOS:263, 265, 267, and 269, which can result in a proline or serine residue at corresponding aa position 158 of SEQ ID NOS:264, 266, 268, and 270; a G/C polymorphism at nt position 753 of SEQ ID NOS:263, 265, 267, and 269, which can result in a glutamine or histidine residue at corresponding aa position 251 of SEQ ID NOS:264, 266, 268, and 270; a silent G/A polymorphism at nt position 1227 of SEQ ID NOS:263, 265, 267, and 269, both of which result in a lysine residue at corresponding aa position 409 of SEQ ID NOS:264, 266, 268, and 270; a silent T/C polymorphism at nt position 1404 of SEQ ID NOS:263, 265, 267, and 269, both of which result in a tyrosine residue at corresponding aa position 468 of SEQ ID NOS:264, 266, 268, and 270; a G/A polymorphism at nt position 2768 of SEQ ID NOS:263, 265, and 269, which can result in an arginine or histidine residue at corresponding aa position 923 of SEQ ID NOS:264, 266, and 270; a T/G polymorphism at nt position 3321 of SEQ ID NOS:263, 265, and 269, which can result in a cysteine or tryptophan residue at corresponding aa position 1107 of SEQ ID NOS:264, 266, and 270; an A/G polymorphism at nt position 4913 of SEQ ID NOS:263, 265, and 269, which can result in a tyrosine or cysteine residue at corresponding aa position 1638 of SEQ ID NOS:264, 266, and 270; a T/C polymorphism at nt position 5264 of SEQ ID NOS:263, 265, and 269, which can result in a phenylalanine or serine residue at corresponding aa position 1755 of SEQ ID NOS:264, 266, and 270; and a G/C polymorphism at nt position 5329 of SEQ ID NOS:263, 265, and 269, which can result in a valine or glutamine residue at corresponding aa position 1777 of SEQ ID NOS:264, 266, and 270. The present invention contemplates sequences comprising any and all combinations and permutations of the above polymorphisms.

An additional application of the described novel human polynucleotide sequences is their use in the molecular mutagenesis/evolution of proteins that are at least partially encoded by the described novel sequences using, for example, polynucleotide shuffling or related methodologies. Such approaches are described in U.S. Pat. Nos. 5,830,721 and 5,837,458.

Therapeutic gene delivery of the described membrane protein nucleotides can be effected by a variety of methods. For example: methods of retroviral human gene therapy are described in, inter alia, U.S. Pat. Nos. 5,399,346 and 5,858,740; adenoviral vectors for gene therapy/delivery are described in U.S. Pat. No. 5,824,544; adeno-associated viral vectors are described in U.S. Pat. Nos. 5,843,742, 5,780,280, and 5,846,528; herpes virus vectors are described in U.S. Pat. No. 5,830,727; and other vectors and methods of non-viral (e.g., polynucleotides that are not encapsulated by viral capsid protein, “naked” DNA, or DNA formulated in lipid or chemical complexes) introduction of foreign genetic material of recombinant origin into host mammalian, and preferably human, cells are described in U.S. Pat. Nos. 5,827,703 and 5,840,710.

The described membrane protein gene products can also be expressed in non-human transgenic animals. Animals of any non-human species, including, but not limited to, worms, mice, rats, rabbits, guinea pigs, pigs, micro-pigs, birds, goats, and non-human primates, e.g., baboons, monkeys, and chimpanzees, may be used to generate membrane protein transgenic animals.

Any technique known in the art may be used to introduce a membrane protein transgene into animals to produce the founder lines of transgenic animals. Such techniques include, but are not limited to, pronuclear microinjection (U.S. Pat. No. 4,873,191), retrovirus-mediated gene transfer into germ lines (Van der Putten et al., Proc. Natl. Acad. Sci. USA 82:6148-6152, 1985), gene targeting in embryonic stem cells (Thompson et al., Cell 56:313-321, 1989), electroporation of embryos (Lo, Mol. Cell. Biol. 3:1803-1814, 1983), and sperm-mediated gene transfer (Lavitrano et al., Cell 57:717-723, 1989), etc. For a review of such techniques, see, e.g., Gordon, Intl. Rev. Cytol. 115:171-229, 1989.

The present invention provides for transgenic animals that carry a membrane protein transgene in all their cells, as well as animals that carry a transgene in some, but not all their cells, i.e., mosaic animals or somatic cell transgenic animals. A transgene may be integrated as a single transgene, or in concatamers, e.g., head-to-head tandems or head-to-tail tandems. A transgene may also be selectively introduced into and activated in a particular cell-type by following, for example, the teaching of Lasko et al., Proc. Natl. Acad. Sci. USA 89:6232-6236, 1992. The regulatory sequences required for such a cell-type specific activation will depend upon the particular cell-type of interest, and will be apparent to those of skill in the art.

When it is desired that a membrane protein transgene be integrated into the chromosomal site of the endogenous membrane protein gene, gene targeting is preferred. Briefly, when such a technique is to be utilized, vectors containing some nucleotide sequences homologous to the endogenous membrane protein gene are designed for the purpose of integrating, via homologous recombination with chromosomal sequences, into and disrupting the function of the nucleotide sequence of the endogenous membrane protein gene (i.e., “knockout” animals).

The transgene can also be selectively introduced into a particular cell-type, thus inactivating the endogenous membrane protein gene in only that cell-type, by following, for example, the teaching of Gu et al., Science 265:103-106, 1994. The regulatory sequences required for such a cell-type specific inactivation will depend upon the particular cell-type of interest, and will be apparent to those of skill in the art.

Once transgenic animals have been generated, the expression of the recombinant membrane protein gene may be assayed utilizing standard techniques. Initial screening may be accomplished by Southern blot analysis or PCR techniques to analyze animal tissues to assay whether integration of the transgene has taken place. The level of mRNA expression of the transgene in the tissues of the transgenic animals may also be assessed using techniques that include, but are not limited to, Northern blot analysis of tissue samples obtained from the animal, in situ hybridization analysis, and RT-PCR. Samples of membrane protein gene-expressing tissue may also be evaluated immunocytochemically using antibodies specific for the membrane protein transgene product.

The present invention also provides for “knock-in” animals. Knock-in animals are those in which a polynucleotide sequence (i.e., a gene or a cDNA) that the animal does not naturally have in its genome is inserted in such a way that it is expressed. Examples include, but are not limited to, a human gene or cDNA used to replace its murine ortholog in the mouse, a murine cDNA used to replace the murine gene in the mouse, and a human gene or cDNA or murine cDNA that is tagged with a reporter construct used to replace the murine ortholog or gene in the mouse. Such replacements can occur at the locus of the murine ortholog or gene, or at another specific site. Such knock-in animals are useful for the in vivo study, testing and validation of, intra alia, human drug targets, as well as for compounds that are directed at the same, and therapeutic proteins.

### 7.2 Membrane Protein Amino Acid Sequences

The described membrane proteins, polypeptides, peptide fragments, mutated, truncated, or deleted forms of the membrane proteins, and/or membrane protein fusion proteins can be prepared for a variety of uses. These uses include, but are not limited to, the generation of antibodies, as reagents in diagnostic assays, for the identification of other cellular gene products related to a membrane protein, and as reagents in assays for screening for compounds that can be used as pharmaceutical reagents useful in the therapeutic treatment of mental, biological, or medical disorders and diseases. Given the similarity information and expression data, the described membrane proteins can be targeted (by drugs, oligonucleotides, antibodies, etc.) in order to diagnose or treat disease, or to therapeutically supplant or augment the efficacy of, for example, chemotherapeutic agents used in the treatment of cancer (for example breast or prostate cancer), therapeutic agents used in the treatment of abnormal blood pressure, heart disease, diabetes, inflammatory disorders, arthritis, Alzheimer's disease, neurodegenerative diseases such as Parkinson's disease, stroke, vascular dementia, conditions requiring modulation of fat and cholesterol metabolism such as coronary artery disease, infectious disease, as antiviral agents, or to promote healing.

The Sequence Listing discloses the amino acid sequences encoded by the described membrane protein nucleic acid sequences. The membrane proteins display initiator methionines in DNA sequence contexts consistent with translation initiation sites, and most contain signal-like sequences often associated with membrane-associated or secreted proteins.

The membrane protein amino acid sequences of the invention include the amino acid sequence presented in the Sequence Listing, as well as analogues and derivatives thereof. Further, corresponding membrane protein homologues from other species are encompassed by the invention. In fact, any membrane protein encoded by the membrane protein nucleotide sequences described herein are within the scope of the invention, as are any novel polynucleotide sequences encoding all or any novel portion of an amino acid sequence presented in the Sequence Listing. The degenerate nature of the genetic code is well-known, and, accordingly, each amino acid presented in the Sequence Listing is generically representative of the well-known nucleic acid “triplet” codon, or in many cases codons, that can encode the amino acid. As such, as contemplated herein, the amino acid sequences presented in the Sequence Listing, when taken together with the genetic code (see, for example, “Molecular Cell Biology”, Table 4-1 at page 109 (Darnell et al., eds., Scientific American Books, New York, N.Y., 1986)), are generically representative of all the various permutations and combinations of nucleic acid sequences that can encode such amino acid sequences.

The invention also encompasses proteins that are functionally equivalent to the membrane proteins encoded by the presently described nucleotide sequences as judged by any of a number of criteria, including, but not limited to, the ability to bind and cleave a substrate of a membrane protein, the ability to effect an identical or complementary downstream pathway, or a change in cellular metabolism (e.g., proteolytic activity, ion flux, tyrosine phosphorylation, etc.). Such functionally equivalent membrane proteins include, but are not limited to, additions or substitutions of amino acid residues within the amino acid sequence encoded by the membrane protein nucleotide sequences described herein, but that result in a silent change, thus producing a functionally equivalent expression product. Amino acid substitutions may be made on the basis of similarity in polarity, charge, solubility, hydrophobicity, hydrophilicity, and/or the amphipathic nature of the residues involved. For example, nonpolar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid.

A variety of host-expression vector systems can be used to express the membrane protein nucleotide sequences of the invention. Where, as in the present instance, the membrane protein, peptide, or polypeptide is thought to be a membrane (or possibly secreted or membrane-associated) protein, the hydrophobic regions of the protein can be excised and the resulting soluble peptide or polypeptide can be recovered from the culture media. Such expression systems also encompass engineered host cells that express a membrane protein, or functional equivalent, in situ. Purification or enrichment of a membrane protein from such expression systems can be accomplished using appropriate detergents and lipid micelles and methods well-known to those skilled in the art. However, such engineered host cells themselves may be used in situations where it is important not only to retain the structural and functional characteristics of a membrane protein, but to assess biological activity, e.g., in certain drug screening assays.

The expression systems that may be used for purposes of the invention include, but are not limited to, microorganisms such as bacteria (e.g., E. coli, B. subtilis) transformed with recombinant bacteriophage DNA, plasmid DNA or cosmid DNA expression vectors containing membrane protein nucleotide sequences; yeast (e.g., Saccharomyces, Pichia) transformed with recombinant yeast expression vectors containing membrane protein nucleotide sequences; insect cell systems infected with recombinant virus expression vectors (e.g., baculovirus) containing membrane protein nucleotide sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing membrane protein nucleotide sequences; or mammalian cell systems (e.g., COS, CHO, BHK, 293, 3T3) harboring recombinant expression constructs containing membrane protein nucleotide sequences and promoters derived from the genome of mammalian cells (e.g., metallothionein promoter) or from mammalian viruses (e.g., the adenovirus late promoter; the vaccinia virus 7.5K promoter).

In bacterial systems, a number of expression vectors may be advantageously selected depending upon the use intended for the membrane protein product being expressed. For example, when a large quantity of such a protein is to be produced for the generation of pharmaceutical compositions of or containing a membrane protein, or for raising antibodies to a membrane protein, vectors that direct the expression of high levels of fusion protein products that are readily purified may be desirable. Such vectors include, but are not limited to, the E. coli expression vector pUR278 (Ruther and Muller-Hill, EMBO J. 2:1791-1794, 1983), in which a membrane protein coding sequence may be ligated individually into the vector in frame with the lacZ coding region so that a fusion protein is produced, pIN vectors (Inouye and Inouye, Nucl. Acids Res. 13:3101-3109, 1985; Van Heeke and Schuster, J. Biol. Chem. 264:5503-5509, 1989), and the like. pGEX vectors (Pharmacia or American Type Culture Collection) can also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. The pGEX vectors are designed to include thrombin or factor Xa protease cleavage sites so that the cloned target expression product can be released from the GST moiety.

In an exemplary insect system, Autographa californica nuclear polyhedrosis virus (AcNPV) is used as a vector to express foreign polynucleotide sequences. The virus grows in Spodoptera frugiperda cells. A membrane protein coding sequence can be cloned individually into a non-essential region (for example the polyhedrin gene) of the virus and placed under control of an AcNPV promoter (for example the polyhedrin promoter). Successful insertion of a membrane protein coding sequence will result in inactivation of the polyhedrin gene and production of non-occluded recombinant virus (i.e., virus lacking the proteinaceous coat coded for by the polyhedrin gene). These recombinant viruses are then used to infect Spodoptera frugiperda cells in which the inserted sequence is expressed (see, e.g., Smith et al., J. Virol. 46:584-593, 1983, and U.S. Pat. No. 4,215,051).

In mammalian host cells, a number of viral-based expression systems may be utilized. In cases where an adenovirus is used as an expression vector, the membrane protein nucleotide sequence of interest may be ligated to an adenovirus transcription/translation control complex, e.g., the late promoter and tripartite leader sequence. This chimeric sequence may then be inserted in the adenovirus genome by in vitro or in vivo recombination. Insertion in a non-essential region of the viral genome (e.g., region E1 or E3) will result in a recombinant virus that is viable and capable of expressing a membrane protein product in infected hosts (see, e.g., Logan and Shenk, Proc. Natl. Acad. Sci. USA 81:3655-3659, 1984). Specific initiation signals may also be required for efficient translation of inserted membrane protein nucleotide sequences. These signals include the ATG initiation codon and adjacent sequences. In cases where an entire membrane protein gene or cDNA, including its own initiation codon and adjacent sequences, is inserted into the appropriate expression vector, no additional translational control signals may be needed. However, in cases where only a portion of a membrane protein coding sequence is inserted, exogenous translational control signals, including, perhaps, the ATG initiation codon, may be provided. Furthermore, the initiation codon should be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see, e.g., Bitter et al., Methods in Enzymol. 153:516-544, 1987).

In addition, a host cell strain may be chosen that modulates the expression of the inserted sequences, or modifies and processes the expression product in the specific fashion desired. Such modifications (e.g., glycosylation) and processing (e.g., cleavage) of protein products may be important for the function of the protein. Different host cells have characteristic and specific mechanisms for the post-translational processing and modification of proteins and expression products. Appropriate cell lines or host systems can be chosen to ensure the desired modification and processing of the foreign protein expressed. To this end, eukaryotic host cells that possess the cellular machinery for the desired processing of the primary transcript, glycosylation, and phosphorylation of the expression product may be used. Such mammalian host cells include, but are not limited to, CHO, VERO, BHK, HeLa, COS, MDCK, 293, 3T3, WI38, and in particular, human cell lines.

For long-term, high-yield production of recombinant proteins, stable expression is preferred. For example, cell lines that stably express the membrane protein sequences described herein can be engineered. Rather than using expression vectors that contain viral origins of replication, host cells can be transformed with DNA controlled by appropriate expression control elements (e.g., promoter, enhancer sequences, transcription terminators, polyadenylation sites, etc.), and a selectable marker. Following the introduction of the foreign DNA, engineered cells may be allowed to grow for 1-2 days in an enriched media, and then switched to a selective media. The selectable marker in the recombinant plasmid confers resistance to the selection and allows cells to stably integrate the plasmid into their chromosomes and grow to form foci, which in turn can be cloned and expanded into cell lines. This method may advantageously be used to engineer cell lines that express a membrane protein product. Such engineered cell lines may be particularly useful in screening and evaluation of compounds that affect the endogenous activity of a membrane protein product.

A number of selection systems may be used, including, but not limited to, the herpes simplex virus thymidine kinase (Wigler et al., Cell 11:223-232, 1977), hypoxanthine-guanine phosphoribosyltransferase (Szybalska and Szybalski, Proc. Natl. Acad. Sci. USA 48:2026-2034, 1962), and adenine phosphoribosyltransferase (Lowy et al., Cell 22:817-823, 1980) genes, which can be employed in tk⁻, hgprt⁻ or aprt⁻ cells, respectively. Also, antimetabolite resistance can be used as the basis of selection for the following genes: dihydrofolate reductase (dhfr), which confers resistance to methotrexate (Wigler et al., Proc. Natl. Acad. Sci. USA 77:3567-3570, 1980, and O'Hare et al., Proc. Natl. Acad. Sci. USA 78:1527-1531, 1981); guanine phosphoribosyl transferase (gpt), which confers resistance to mycophenolic acid (Mulligan and Berg, Proc. Natl. Acad. Sci. USA 78:2072-2076, 1981); neomycin phosphotransferase (neo), which confers resistance to G-418 (Colbere-Garapin et al., J. Mol. Biol. 150:1-14, 1981); and hygromycin B phosphotransferase (hpt), which confers resistance to hygromycin (Santerre et al., Gene 30:147-156, 1984).

Alternatively, any fusion protein can be readily purified by utilizing an antibody specific for the fusion protein being expressed. Another exemplary system allows for the ready purification of non-denatured fusion proteins expressed in human cell lines (Janknecht et al., Proc. Natl. Acad. Sci. USA 88:8972-8976, 1991). In this system, the sequence of interest is subcloned into a vaccinia recombination plasmid such that the sequence's open reading frame is translationally fused to an amino-terminal tag consisting of six histidine residues. Extracts from cells infected with recombinant vaccinia virus are loaded onto Ni²⁺•nitriloacetic acid-agarose columns, and histidine-tagged proteins are selectively eluted with imidazole-containing buffers.

Also encompassed by the present invention are fusion proteins that direct a membrane protein to a target organ and/or facilitate transport across the membrane into the cytosol. Conjugation of membrane proteins to antibody molecules or their Fab fragments could be used to target cells bearing a particular epitope. Attaching an appropriate signal sequence to a membrane protein would also transport a membrane protein to a desired location within the cell. Alternatively targeting of a membrane protein or its nucleic acid sequence might be achieved using liposome or lipid complex based delivery systems. Such technologies are described in “Liposomes: A Practical Approach” (New, R. R. C., ed., IRL Press, New York, N.Y., 1990), and in U.S. Pat. Nos. 4,594,595, 5,459,127, 5,948,767 and 6,110,490. Additionally embodied are novel protein constructs engineered in such a way that they facilitate transport of membrane proteins to a target site or desired organ, where they cross the cell membrane and/or the nucleus where the membrane proteins can exert their functional activity. This goal may be achieved by coupling of a membrane protein to a cytokine or other ligand that provides targeting specificity, and/or to a protein transducing domain (see generally U.S. Provisional Patent Application Ser. Nos. 60/111,701 and 60/056,713 for examples of such transducing sequences), to facilitate passage across cellular membranes, and can optionally be engineered to include nuclear localization signals.

Additionally contemplated are oligopeptides that are modeled on an amino acid sequence first described in the Sequence Listing. Such membrane protein oligopeptides are generally between about 10 to about 100 amino acids long, or between about 16 to about 80 amino acids long, or between about 20 to about 35 amino acids long, or any variation or combination of sizes represented therein that incorporate a contiguous region of sequence first disclosed in the Sequence Listing. Such membrane protein oligopeptides can be of any length disclosed within the above ranges and can initiate at any amino acid position represented in the Sequence Listing.

The invention also contemplates “substantially isolated” or “substantially pure” proteins or polypeptides. By a “substantially isolated” or “substantially pure” protein or polypeptide is meant a protein or polypeptide that has been separated from at least some of those components that naturally accompany it. Typically, the protein or polypeptide is substantially isolated or pure when it is at least 60%, by weight, free from the proteins and other naturally-occurring organic molecules with which it is naturally associated in vivo. Preferably, the purity of the preparation is at least 75%, more preferably at least 90%, and most preferably at least 99%, by weight. A substantially isolated or pure protein or polypeptide may be obtained, for example, by extraction from a natural source, by expression of a recombinant nucleic acid encoding the protein or polypeptide, or by chemically synthesizing the protein or polypeptide.

Purity can be measured by any appropriate method, e.g., column chromatography such as immunoaffinity chromatography using an antibody specific for the protein or polypeptide, polyacrylamide gel electrophoresis, or HPLC analysis. A protein or polypeptide is substantially free of naturally associated components when it is separated from at least some of those contaminants that accompany it in its natural state. Thus, a polypeptide that is chemically synthesized or produced in a cellular system different from the cell from which it naturally originates will be, by definition, substantially free from its naturally associated components. Accordingly, substantially isolated or pure proteins or polypeptides include eukaryotic proteins synthesized in E. coli, other prokaryotes, or any other organism in which they do not naturally occur.

### 7.3 Membrane Protein Antibodies

Antibodies that specifically recognize one or more epitopes of a membrane protein, epitopes of conserved variants of a membrane protein, or peptide fragments of a membrane protein, are also encompassed by the invention. Such antibodies include, but are not limited to, polyclonal antibodies, monoclonal antibodies (mAbs), humanized or chimeric antibodies, single chain antibodies, Fab fragments, F(ab′)₂ fragments, fragments produced by a Fab expression library, anti-idiotypic (anti-Id) antibodies, and epitope-binding fragments of any of the above.

The antibodies of the invention may be used, for example, in the detection of a membrane protein in a biological sample and may, therefore, be utilized as part of a diagnostic or prognostic technique whereby patients may be tested for abnormal amounts of a membrane protein. Such antibodies may also be utilized in conjunction with, for example, compound screening schemes for the evaluation of the effect of test compounds on expression and/or activity of a membrane protein expression product. Additionally, such antibodies can be used in conjunction with gene therapy to, for example, evaluate normal and/or engineered membrane protein-expressing cells prior to their introduction into a patient. Such antibodies may additionally be used in methods for the inhibition of abnormal membrane protein activity. Thus, such antibodies may be utilized as a part of treatment methods.

For the production of antibodies, various host animals may be immunized by injection with a membrane protein, peptide (e.g., one corresponding to a functional domain of a membrane protein), truncated membrane protein polypeptide (a membrane protein in which one or more domains have been deleted), functional equivalents of a membrane protein, or mutated variants of a membrane protein. Such host animals may include, but are not limited to, pigs, rabbits, mice, goats, and rats, to name but a few. Various adjuvants may be used to increase the immunological response, depending on the host species, including, but not limited to, Freund's adjuvant (complete and incomplete), mineral salts such as aluminum hydroxide or aluminum phosphate, chitosan, surface active substances such as lysolecithin, pluronic polyols, polyanions, peptides, oil emulsions, and potentially useful human adjuvants such as BCG (bacille Calmette-Guerin) and Corynebacterium parvum. Alternatively, the immune response could be enhanced by combination and/or coupling with molecules such as keyhole limpet hemocyanin, tetanus toxoid, diphtheria toxoid, ovalbumin, cholera toxin, or fragments thereof. Polyclonal antibodies are heterogeneous populations of antibody molecules derived from the sera of the immunized animals.

Monoclonal antibodies, which are homogeneous populations of antibodies to a particular antigen, can be obtained by any technique that provides for the production of antibody molecules by continuous cell lines in culture. These include, but are not limited to, the hybridoma technique (Kohler and Milstein, Nature 256:495-497, 1975, and U.S. Pat. No. 4,376,110), the human B-cell hybridoma technique (Kosbor et al., Immunology Today 4:72, 1983, and Cole et al., Proc. Natl. Acad. Sci. USA 80:2026-2030, 1983), and the EBV-hybridoma technique (Cole et al., in “Monoclonal Antibodies and Cancer Therapy”, Vol. 27, UCLA Symposia on Molecular and Cellular Biology, New Series, pp. 77-96 (Reisfeld and Sell, eds., Alan R. Liss, Inc. New York, N.Y., 1985)). Such antibodies may be of any immunoglobulin class, including IgG, IgM, IgE, IgA, and IgD, and any subclass thereof. The hybridomas producing the mAbs of this invention may be cultivated in vitro or in vivo. Production of high titers of mAbs in vivo makes this the presently preferred method of production.

In addition, techniques developed for the production of “chimeric antibodies” (Morrison et al., Proc. Natl. Acad. Sci. USA 81:6851-6855, 1984, Neuberger et al., Nature 312:604-608, 1984, and Takeda et al., Nature 314:452-454, 1985) by splicing the genes from a mouse antibody molecule of appropriate antigen specificity together with genes from a human antibody molecule of appropriate biological activity can be used. A chimeric antibody is a molecule in which different portions are derived from different animal species, such as those having a variable region derived from a murine mAb and a human immunoglobulin constant region. Such technologies are described in U.S. Pat. Nos. 6,114,598, 6,075,181 and 5,877,397. Also encompassed by the present invention is the use of fully humanized monoclonal antibodies, as described in U.S. Pat. No. 6,150,584.

Alternatively, techniques described for the production of single chain antibodies (U.S. Pat. No. 4,946,778, Bird, Science 242:423-426, 1988, Huston et al., Proc. Natl. Acad. Sci. USA 85:5879-5883, 1988, and Ward et al., Nature 341:544-546, 1989) can be adapted to produce single chain antibodies against membrane protein expression products. Single chain antibodies are formed by linking the heavy and light chain fragments of the Fv region via an amino acid bridge, resulting in a single chain polypeptide.

Antibody fragments that recognize specific epitopes may be generated by known techniques. For example, such fragments include, but are not limited to: F(ab′)₂ fragments, which can be produced by pepsin digestion of an antibody molecule; and Fab fragments, which can be generated by reducing the disulfide bridges of F(ab′)₂ fragments. Alternatively, Fab expression libraries may be constructed (Huse et al., Science 246:1275-1281, 1989) to allow rapid and easy identification of monoclonal Fab fragments with the desired specificity.

Antibodies to a membrane protein can, in turn, be utilized to generate anti-idiotype antibodies that “mimic” a given membrane protein, using techniques well-known to those skilled in the art (see, e.g., Greenspan and Bona, FASEB J. 7:437-444, 1993, and Nissinoff, J. Immunol. 147:2429-2438, 1991). For example, antibodies that bind to a membrane protein domain and competitively inhibit the binding of a membrane protein to its cognate receptor can be used to generate anti-idiotypes that “mimic” the membrane protein and, therefore, bind and activate or neutralize a receptor. Such anti-idiotypic antibodies, or Fab fragments of such anti-idiotypes, can be used in therapeutic regimens involving a membrane protein-mediated pathway.

Additionally given the high degree of relatedness of mammalian membrane proteins, membrane protein knock-out mice (having never seen a membrane protein, and thus never been tolerized to a membrane protein) have an unique utility, as they can be advantageously applied to the generation of antibodies against the disclosed mammalian membrane proteins (i.e., a membrane protein will be immunogenic in the corresponding membrane protein knock-out animals).

The present invention is not to be limited in scope by the specific embodiments described herein, which are intended as single illustrations of individual aspects of the invention, and functionally equivalent methods and components are within the scope of the invention. Indeed, various modifications of the invention, in addition to those shown and described herein, will become apparent to those skilled in the art from the foregoing description. Such modifications are intended to fall within the scope of the appended claims. All cited publications, patents, and patent applications are herein incorporated by reference in their entirety.

1. An isolated nucleic acid molecule comprising a nucleotide sequence that encodes the amino acid sequence of SEQ ID NO: 2, 4, 6, 8, 10, 12, 14, 16, 18, 20, 22, 24, 26, 28, 30, 33, 35, 37, 39, 41, 43, 45, 47, 49, 51, 53, 55, 57, 59, 61, 64, 66, 68, 70, 72, 74, 76, 78, 80, 82, 84, 86, 88, 90, 93, 95, 97, 99, 101, 103, 105, 107, 109, 111, 114, 116, 119, 121, 123, 126, 128, 130, 132, 134, 136, 138, 140, 142, 144, 146, 148, 150, 152, 154, 156, 159, 161, 163, 165, 167, 169, 171, 173, 175, 177, 179, 181, 183, 185, 187, 189, 191, 194, 196, 198, 200, 202, 205, 207, 209, 211, 213, 216, 218, 220, 223, 225, 227, 230, 232, 235, 238, 240, 242, 244, 246, 248, 250, 252, 254, 256, 258, 260, 262, 264, 266, 268, or 270.

2. The isolated nucleic acid molecule of claim 1, wherein said nucleic acid molecule comprises the nucleotide sequence of SEQ ID NO: 1, 3, 5, 7, 9, 1, 13, 15, 17, 19, 21, 23, 25, 27, 29, 32, 34, 36, 38, 40, 42, 44, 46, 48, 50, 52, 54, 56, 58, 60, 63, 65, 67, 69, 71, 73, 75, 77, 79, 81, 83, 85, 87, 89, 92, 94, 96, 98, 100, 102, 104, 106, 108, 110, 113, 115, 118, 120, 122, 125, 127, 129, 131, 133, 135, 137, 139, 141, 143, 145, 147, 149, 151, 153, 155, 158, 160, 162, 164, 166, 168, 170, 172, 174, 176, 178, 180, 182, 184, 186, 188, 190, 193, 195, 197, 199, 201, 204, 206, 208, 210, 212, 215, 217, 219, 222, 224, 226, 229, 231, 234, 237, 239, 241, 243, 245, 247, 249, 251, 253, 255, 257, 259, 261, 263, 265, 267, or 269.

3. An expression vector comprising the isolated nucleic acid molecule of claim 1.

4. An isolated polypeptide comprising the amino acid sequence of SEQ ID NO: 2, 4, 6, 8, 10, 12, 14, 16, 18, 20, 22, 24, 26, 28, 30, 33, 35, 37, 39, 41, 43, 45, 47, 49, 51, 53, 55, 57, 59, 61, 64, 66, 68, 70, 72, 74, 76, 78, 80, 82, 84, 86, 88, 90, 93, 95, 97, 99, 101, 103, 105, 107, 109, 111, 114, 116, 119, 121, 123, 126, 128, 130, 132, 134, 136, 138, 140, 142, 144, 146, 148, 150, 152, 154, 156, 159, 161, 163, 165, 167, 169, 171, 173, 175, 177, 179, 181, 183, 185, 187, 189, 191, 194, 196, 198, 200, 202, 205, 207, 209, 211, 213, 216, 218, 220, 223, 225, 227, 230, 232, 235, 238, 240, 242, 244, 246, 248, 250, 252, 254, 256, 258, 260, 262, 264, 266, 268, or 270.

5. An antibody that selectively binds a polypeptide drawn from the group consisting of: SEQ ID NO: 2, 4, 6, 8, 10, 12, 14, 16, 18, 20, 22, 24, 26, 28, 30, 33, 35, 37, 39, 41, 43, 45, 47, 49, 51, 53, 55, 57, 59, 61, 64, 66, 68, 70, 72, 74, 76, 78, 80, 82, 84, 86, 88, 90, 93, 95, 97, 99, 101, 103, 105, 107, 109, 111, 114, 116, 119, 121, 123, 126, 128, 130, 132, 134, 136, 138, 140, 142, 144, 146, 148, 150, 152, 154, 156, 159, 161, 163, 165, 167, 169, 171, 173, 175, 177, 179, 181, 183, 185, 187, 189, 191, 194, 196, 198, 200, 202, 205, 207, 209, 211, 213, 216, 218, 220, 223, 225, 227, 230, 232, 235, 238, 240, 242, 244, 246, 248, 250, 252, 254, 256, 258, 260, 262, 264, 266, 268, or 270.

6. An oligonucleotide that inhibits the expression of a nucleic acid molecule that encodes an amino acid sequence drawn from the group consisting of: SEQ ID NO: 2, 4, 6, 8, 10, 12, 14, 16, 18, 20, 22, 24, 26, 28, 30, 33, 35, 37, 39, 41, 43, 45, 47, 49, 51, 53, 55, 57, 59, 61, 64, 66, 68, 70, 72, 74, 76, 78, 80, 82, 84, 86, 88, 90, 93, 95, 97, 99, 101, 103, 105, 107, 109, 111, 114, 116, 119, 121, 123, 126, 128, 130, 132, 134, 136, 138, 140, 142, 144, 146, 148, 150, 152, 154, 156, 159, 161, 163, 165, 167, 169, 171, 173, 175, 177, 179, 181, 183, 185, 187, 189, 191, 194, 196, 198, 200, 202, 205, 207, 209, 211, 213, 216, 218, 220, 223, 225, 227, 230, 232, 235, 238, 240, 242, 244, 246, 248, 250, 252, 254, 256, 258, 260, 262, 264, 266, 268, or 270.
